(19) United States
(12) Patent Application Publication
(10) Pub. No.: US 2022/0401085 A1
(43) Pub. Date: Dec. 22, 2022

(54) ELECTROSURGICAL SYSTEM

(71) Applicant: Eric R. Cosman, JR., Cambridge, MA (US)

(72) Inventor: Eric R. Cosman, Jr., Cambridge, MA (US)

(73) Assignee: Eric R. Cosman, JR., Cambridge, MA (US)

(21) Appl. No.: 17/779,409

(22) PCT Filed: Nov. 25, 2020
(86) PCT No.: PCT/US2020/062443

§ 371 Date: May 24, 2022

Related U.S. Application Data
(60) Provisional application No. 63/115,028, filed on Nov. 18, 2020.
(60) Provisional application No. 63/024,485, filed on May 13, 2020.
(60) Provisional application No. 62/948,284, filed on Dec. 15, 2019.
(60) Provisional application No. 62/940,222, filed on Nov. 25, 2019.

(51) Int. Cl. A61B 10/02; A61B 18/14

(52) U.S. Cl. CPC A61B 10/0266; A61B 18/1477; A61B 2018/00577

(57) ABSTRACT

This invention relates to electrosurgical systems for coagulation and ablation of tissue, including in relation to tissue biopsy.

### CLAIM OF PRIORITY

This application claims the benefit of U.S. Provisional Application No. 63/115,028, filed on Nov. 18, 2020, U.S. Provisional Application No. 63/024,485, filed on May 13, 2020, U.S. Provisional Application No. 62/948,284, filed on Dec. 15, 2020, and U.S. Provisional Application No. 62/940,222, filed on Nov. 25, 2019, each of which is incorporated by reference in its entirety.

### TECHNICAL FIELD

This invention relates to electrosurgical systems.

### BACKGROUND

Biopsy needles, including without limitation core biopsy needles and fine-needle aspiration needles, are inserted into bodily tissue (such as that of a human patient or an animal) for the purpose of collecting a tissue sample from a target structure (such as tumor or suspected tumor) in the bodily tissue. In some procedures, a biopsy needle is inserted into the bodily tissue through an introducer cannula which is first inserted into the bodily tissue. Such insertions can cause bleeding within the bodily tissue around the tract formed by the insertion of the biopsy needle. Biopsy can cause tumor seeding, wherein contact between tumor cells and a biopsy needle or the biopsy needle introducer causes the spread of the tumor cells into healthy bodily tissue when the biopsy needle or its introducer are withdrawn from the bodily tissue because such tumor cells can adhere to the biopsy needle or its introducer and be dragged into more superficial tissue along the tissue tract formed by the biopsy needle or its introducer. Such bleeding and tumor seeding can be an undesirable adverse event due to tissue biopsy.

An electrosurgical probe, such as a radiofrequency (RF) electrode or a microwave (MW) antenna, can be used to ablate tissue around the probe when inserted into tissue by delivering electrical energy to the tissue, thereby heating the tissue to a destructive temperature and coagulating the tissue and blood in the tissue. Monopolar ablation (such as monopolar RF ablation) refers to an RF electrosurgical configuration in which current from the ablation probe is conducted back the electrosurgical generator (such as an RF generator) by a conductive plate electrode (also known as a “ground pad” or “ground plate”) placed in contact with the skin of the bodily tissue. Bipolar ablation (such as bipolar RF ablation) refers an RF electrosurgical configuration in which two electrically-conductive contacts of one or more ablation probes are at two different electrical potentials (also known as “poles” of an electrosurgical generator) such that electrical current flows between the two contacts; for example a bipolar electrode can have two electrical contacts on a single shaft that are inserted into tissue. Electrodes, needles, antenna, and other elongated medical probes can be said to have a “distal” end that is advanced into tissue, and a “proximal end” that is at the opposite end of the elongated probe.

### SUMMARY

There is a need to control bleeding and or tumor seeding due to tissue biopsy. There is a need to coagulate tissue around the distal opening of the introducer of a biopsy needle in order to limit tumor seeding, because tumor cells adhering to the biopsy needle can be dragged up to a deposit at or around the introducer's distal opening when the biopsy needle is withdrawn from the introducer to collect a tissue sample. There is a need to facilitate biopsy of tissue that is about to be ablated. There is a need to reduce the number of tissue tracts formed in tissue when biopsying tissue that is about to be ablated. There is a needle for bipolar electrodes that effectively coagulate tissue. There is a need for electrode and cannula systems that have simple construction and that effectively ablate tissue. In one aspect, the present invention relates to the electrosurgical ablation of the tissue tract (which can be referred to as “tract burn” or “track burn”) created by a biopsy needle and/or its introducer for the purpose of limiting bleeding and/or tumor seeding due to the insertion of the biopsy needle and/or its introducer. In one aspect, the present invention relates to a cannula and a probe configured to coagulate and/or ablate tissue around the tract formed by a biopsy needle that was inserted through the cannula and at the distal opening of the cannula from which the biopsy needle extended into the tissue. In one aspect, the present invention relates to the use of an cannula and a probe to coagulate and/or ablate tissue around the tract formed by a biopsy needle that was inserted through the cannula and around the distal opening of the cannula from which the biopsy needle inserted into the tissue, wherein the cannula includes an electrically-insulated proximal shaft portion and an electrically-conductive distal shaft portion. In some embodiments of the present invention, the probe is an electrode. In some embodiments, the cannula is an electrode. In some embodiments, the probe is the biopsy needle. In some embodiments, the combination of the cannula and probe operate as a monopolar RF electrode. In some embodiments, the combination of the cannula and probe operate as a bipolar RF electrode. In one aspect, the present invention relates to construction of monopolar and bipolar electrode systems for tissue ablation and coagulation.

In one aspect, a system can include a cannula and an electrode, wherein the cannula is configured to introduce a biopsy needle into bodily tissue, the cannula is configured to introduce the electrode into the bodily tissue, the cannula shaft includes an electrically conductive shaft portion, the electrode shaft includes an electrically conductive shaft portion, the combination of the cannula and electrode is configured to operate with an electrosurgical generator to ablate tissue around a tract through the tissue formed by prior introduction of the biopsy needle through the cannula and around the cannula distal end.

In certain circumstances, the cannula shaft can include an electrically insulated portion at its proximal end, the cannula shaft includes an electrically conductive portion at its distal end around which tissue is ablated.

In certain circumstances, the biopsy needle can include the electrode.

In certain circumstances, the electrode and the biopsy needle can protrude into the tissue beyond the cannula by the same length when each of the electrode and the biopsy needle are introduced by the cannula.

In certain circumstances, the electrode can be monopolar and cannula conductive shaft portion can be energized by the electrode when the electrode introduced into the tissue via the cannula.

In certain circumstances, the electrode can be bipolar and cannula conductive shaft portion can be energized by one of the electrodes contacts when the electrode when the electrode introduced into the tissue via the cannula.

In certain circumstances, the electrode can be a bipolar electrode.

In certain circumstances, the distal contact of the electrode can have smaller surface area than does the proximal contact of the electrode.

In certain circumstances, the electrode can be a bipolar electrode, wherein the distal contact of the electrode can have smaller surface area than does the proximal contact of the electrode, and wherein the cannula shaft conductive portion can have smaller surface area than the distal contact of the electrode.

In certain circumstances, the system can further include a stylet, wherein the cannula can include the electrode, the cannula can be configured to introduce the stylet into bodily tissue, and tissue can be ablated around the stylet.

Other aspects, embodiments, and features will be apparent from the following description, the drawings, and the claims.

### BRIEF DESCRIPTION OF THE DRAWINGS

FIG. 1A is a schematic drawing of a monopolar electrosurgical system configuration in which a probe includes both biopsy and ablation-electrode functions and electrifies itself and the electrically conductive distal portion of its introducer cannula, whose shaft also includes an electrically insulated proximal portion, to coagulate the tissue tract formed by the biopsy probe, including the tissue around the cannula distal opening through which the biopsy probe extended into the tissue.

FIG. 1B is a schematic drawing of a monopolar electrosurgical system configuration in which a probe includes both biopsy and cooled-ablation-electrode functions and is used to biopsy tissue immediately before ablating the tissue.

FIG. 1C is a schematic drawing of the components of the systems of FIGS. 1A and 1B.

FIG. 1D is a schematic drawing of the components of the systems of FIGS. 1A and 1B.

FIG. 2A is a schematic drawing of a bipolar electrosurgical system configuration in which a probe includes both biopsy and ablation-electrode functions and electrifies itself and the electrically conductive distal portion of its introducer cannula, whose shaft also includes an electrically insulated proximal portion, to coagulate the tissue tract formed by the probe, including the tissue around the cannula distal opening through which the probe extended into the tissue.

FIG. 2B is a schematic drawing of the components of the system of FIG. 2A.

FIG. 3 is a schematic drawing of an electrode surgical system that include a biopsy needle, an electrode, and a cannula having a proximally electrically-non-conductive shaft portion and a distal electrical-conductive shaft portion; the system being configured to provide for biopsy and ablation of tissue.

FIGS. 4A-4D are schematic drawings of a system in which tissue biopsy is performed and then then tissue tract formed due to the biopsy is coagulated.

FIG. 5A is a schematic drawing of a bipolar electrosurgical system that includes a bipolar electrode and a partially electrically insulated cannula, wherein the electrode electrifies the cannula electrically conductive shaft portion of the cannula by means of proximal electrode electrically conductive shaft portion.

FIG. 5B is a schematic drawing of the components of the system of FIG. 5A.

FIGS. 6A-6G are schematic drawings of an electrosurgical system including a cannula having electrically insulated proximal and electrically uninsulated distal shaft portions, an electrically uninsulated monopolar electrode, and a biopsy needle, wherein the electrode and biopsy needles are configured to be inserted into tissue by extending out of the cannula by substantially similar lengths, whereby the tissue tract formed insertion of the biopsy needle can be coagulated, including the portion around the cannula opening from the which the biopsy needle extends into tissue.

FIGS. 7A-7F are schematic drawings of an electrosurgical system including a cannula having electrically insulated proximal and electrically uninsulated distal shaft portions, a monopolar electrode having electrically insulated proximal and electrically uninsulated distal shaft portions, and a biopsy needle, wherein the electrode and biopsy needles are configured to be inserted into tissue by extending out of the cannula by substantially similar lengths, whereby the tissue tract formed insertion of the biopsy needle can be coagulated, including the portion around the cannula opening from the which the biopsy needle extends into tissue.

FIGS. 8A-8G are schematic drawings of an electrosurgical system including a cannula having electrically insulated proximal and electrically uninsulated distal shaft portions, a bipolar electrode of whom one contact is an electrically conductive distal shaft portion and of whom the opposite contact is an electrically conductive proximal shaft portion, and a biopsy needle, wherein the electrode and biopsy needles are configured to be inserted into tissue by extending out of the cannula by substantially similar lengths, whereby the tissue tract formed insertion of the biopsy needle can be coagulated, including the portion around the cannula opening from the which the biopsy needle extends into tissue.

FIGS. 9A-9G are schematic drawings of an electrosurgical system including a cannula having electrically insulated proximal and electrically uninsulated distal shaft portions; a bipolar electrode of whom one contact is an electrically conductive distal shaft portion, of whom the opposite contact is an electrically conductive more proximal shaft portion, and whose proximal shaft portion is electrically insulated; and a biopsy needle; wherein the electrode and biopsy needles are configured to be inserted into tissue by extending out of the cannula by substantially similar lengths; whereby the tissue tract formed insertion of the biopsy needle can be coagulated, including the portion around the cannula opening from the which the biopsy needle extends into tissue.

FIG. 10A is a schematic drawing of a cannula whose shaft is wholly electrically conductive.

FIGS. 10B, 10C, 10D, and 10E are schematic drawings in which the cannula of FIG. 10A replaces the cannula of the systems of FIGS. 6A-6G, 7A-7F, 8A-8G, 9A-9G, respectively.

FIG. 11 a schematic drawing of a monopolar ablation system in which a monopolar electrode is introduced into tissue by a cannula.

FIG. 12 a schematic drawing of a bipolar ablation system in which a bipolar electrode is introduced into tissue by a cannula.

FIGS. 13A, 13B, 13C, 13D are schematic drawings of wherein a bipolar electrode is used to coagulate the tissue tract formed by insertion of a biopsy needle through a cannula by means of withdrawal of the electrode into the cannula.

FIG. 14 a schematic drawing of an electrosurgical system including a biopsy needle, a bipolar electrode, a monopolar electrode, a spacer, and an introducer cannula.

FIGS. 15A, 15B, 15C, 15D are schematic drawings of wherein a bipolar electrode is used to coagulate the tissue tract formed by insertion of a biopsy needle through a cannula by means of withdrawal of the combination of the electrode and cannula.

FIG. 16A is a schematic drawing of a cannula whose shaft is wholly uninsulated and connectable to a pole of an electrosurgical generator, an electrically conductive stylet having a sharp distal end, and a monopolar electrode whose distal conductive end is connectable to a pole of an electrosurgical generator and whose proximal conductive end is configured to be electrified by the cannula when the electrode is inserted into the cannula, wherein these items are configured to ablate the tissue tract formed by a biopsy needle introduced via the cannula.

FIG. 16B is a schematic drawing showing a monopolar coagulation of a biopsy needle tissue tract by means of (1) an uninsulated cannula connected to an electrosurgical generator and (2) a conductive stylet that is electrified by insertion into the cannula.

FIG. 16C is a schematic drawing showing a bipolar coagulation of a biopsy needle tissue tract by means of (1) an uninsulated cannula connected to one pole of an electrosurgical generator and (2) an electrode whose distal contact is connected to the other pole of the electrosurgical generator, and whose proximal conductive shaft is electrified by insertion into the cannula.

FIG. 16D is a schematic drawing showing a bipolar coagulation of a biopsy needle tissue tract by means of (1) an uninsulated cannula connected to one pole of an electrosurgical generator and (2) an electrode whose distal contact is connected to the other pole of the electrosurgical generator, and whose proximal shaft is electrically insulated.

FIG. 17A is a schematic drawing of a cannula whose proximal shaft is electrically insulated and whose distal shaft is electrically conductive and connectable to a pole of an electrosurgical generator, and an electrically conductive stylet having a blunt distal end, wherein these items are configured to ablate the tissue tract formed by a biopsy needle introduced via the cannula.

FIG. 17B is a schematic drawing showing a monopolar coagulation of a biopsy needle tissue tract by means of (1) a partially electrically insulated cannula connected to an electrosurgical generator and (2) a conductive stylet that is electrified by insertion into the cannula.

FIG. 17C is a schematic drawing showing a bipolar coagulation of a biopsy needle tissue tract by means of (1) a partially electrically insulated cannula connected to one pole of an electrosurgical generator and (2) an electrode whose distal contact is connected to the other pole of the electrosurgical generator, and whose proximal conductive shaft is electrified by insertion into the cannula.

FIG. 17D is a schematic drawing showing a bipolar coagulation of a biopsy needle tissue tract by means of (1) a partially electrically insulated cannula connected to one pole of an electrosurgical generator and (2) an electrode whose distal contact is connected to the other pole of the electrosurgical generator, and whose proximal shaft is electrically insulated.

FIG. 18A is a schematic drawing of a cannula that is a bipolar electrode connectable to both poles of an electrosurgical generator, and of a biopsy needle, wherein the cannula is configured to ablate the tissue tract formed by the biopsy needle after the biopsy needle has been introduced via the cannula to collect tissue samples.

FIG. 18B is a schematic drawing showing a bipolar coagulation of a biopsy needle tissue tract by means of (1) a bipolar cannula whose two electrical contacts are connected to opposite poles of an electrosurgical generator and (2) an electrically conductive biopsy needle whose shaft protrudes from the cannula into tissue and which is electrified by its insertion into the cannula.

FIG. 19A is a schematic drawing of a cannula that is a bipolar electrode connectable to both poles of an electrosurgical generator, and of a biopsy needle, wherein one of the cannula's electrical contacts forms the inner lumen of the cannula and is shrouded by electrical insulation, and wherein the cannula is configured to ablate the tissue tract formed by the biopsy needle after the biopsy needle has been introduced via the cannula to collect tissue samples.

FIG. 19B is a schematic drawing showing a bipolar coagulation of a biopsy needle tissue tract by means of (1) a bipolar cannula whose two electrical contacts are connected to opposite poles of an electrosurgical generator and (2) an electrically conductive biopsy needle whose shaft protrudes from the cannula into tissue and which is electrified by its insertion into the cannula and by its physical contact with the cannula's electrical contact that forms cannula's inner lumen.

FIG. 19C is a schematic drawing of a longitudinal cross section of the cannula of FIGS. 19A and 19B.

### DETAILED DESCRIPTION

In one aspect, this invention pertains to the use of tissue coagulation (also known as tissue ablation) in coordination with tissue biopsy, including, without limitation, to control bleeding as a result of tissue biopsy to limit spread of tumor cells due to biopsy (also known as “tumor seeding”), and to reduce the number of penetrations of tissue required to biopsy tissue and then to ablate that tissue. Tissue ablation can be performed by means of radiofrequency (RF) generator output, microwave (MW) generator output, or another type of high frequency electrical signal generator output. RF generators and RF electrodes have the advantage of being low cost; allowing for impedance monitoring, control, and user display; allowing for temperature monitoring, control, and user display.

Some embodiments of the present invention include an ablation electrode (e.g. 120, 220, or 320) that is introduced by an insulated cannula (e.g. 150) and that is used cooperatively with said cannula to coagulate tissue (also known as “ablate tissue”). In some embodiments the electrode is also a biopsy device (e.g. 120 or 220 or 520). In some embodiments, the electrode (e.g. 320) is compatible with a separate biopsy device (e.g. 370); for example, wherein the biopsy device is a widely-used biopsy device provided separately or marketed by a different company; wherein examples of compatibility include that the electrode fits through the same coaxial introducer cannula (e.g. 150) as the biopsy device and extends beyond said introducer cannula by similar, equal, somewhat greater, or greater length, for example, such that the electrode follows and/or ablates the tissue tract made by the biopsy device. Some embodiments of the present invention include an electrosurgical generator 100 (e.g. a radiofrequency generator) having output level (e.g. current, power, voltage) and/or temperature and/or impedance displays (e.g. numeric, time-dependent line graphs, dynamic bar graphs, dynamic graphs showing the current and a minimum and/or maximum value, dynamic meters, and other graphical displays) for an ablation electrode. In one method of the present invention:
An introducer cannula (e.g. 150 or 550), whose proximal shaft is electrically-insulated and whose distal shaft is electrically conductive, is inserted into bodily tissue (e.g. 160). A stylet is optionally used to obdurate said cannula during said insertion, and is removed after said insertion.
A biopsy device (e.g. 120, 220, 370, or 520) is introduced through the introducer (e.g. 150 or 550) and a sample of the target tissue 169 (e.g. a suspected tumor, or a confirmed tumor) is collected using the biopsy device.
Either (a) the biopsy device (e.g. 370) is removed from the introducer and replaced by an ablation electrode (e.g. 320) through the introducer to a position in the tissue substantially similar to, or that includes, the biopsy device's position in the tissue during collection of the tissue sample; or (b) the biopsy device (e.g. 120, 220, or 520) is also an ablation electrode and is replaced (or left remain) in the introducer, in a position in the tissue that is substantially similar to, or includes, the biopsy device's position in the tissue during collection of the tissue sample; wherein the ablation electrode has an electrically-conductive portion at or near the distal end of its shaft.
The electrode (e.g. 120, 220, 320, or 520) is connected to the high-frequency electrical output of a generator (e.g. 100; which can be RF, MW or another high-frequency electrical output) such that the tissue is ablated around electrically-conductive portion of the electrode shaft electrically-conductive portion of the cannula shaft.
The generator (e.g. 100) displays at least one measurement related to tissue heating near the ablation electrode (e.g. temperature, impedance, current, power, voltage).

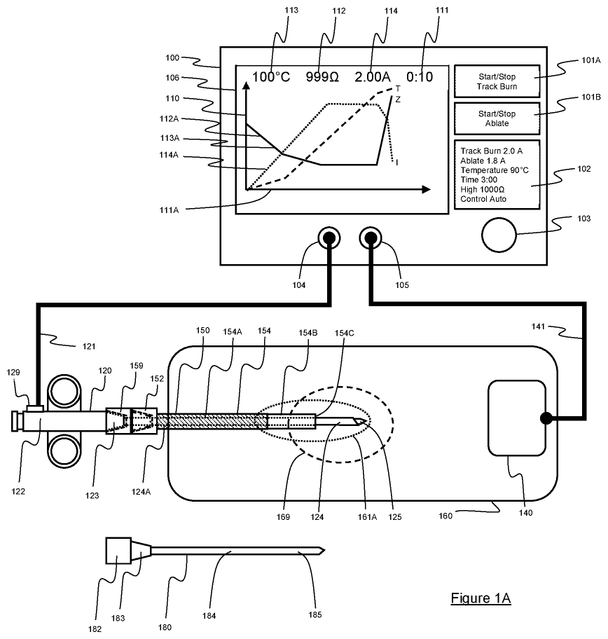

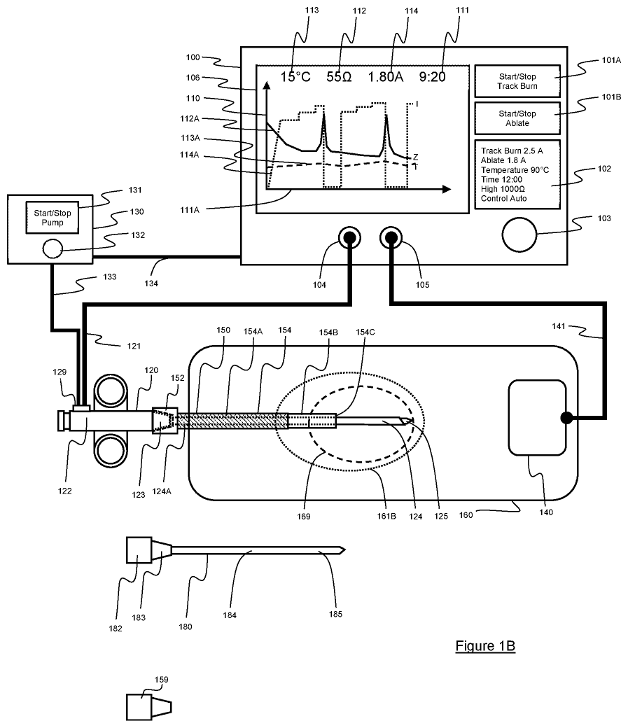

FIG. 1 refers collectively to FIGS. 1A, 1B, 1C, 1D.

FIG. 1A shows one embodiment of the present invention that includes an RF generator connected to a core biopsy device 120 that is also a monopolar ablation electrode 120 and that is inserted into body 160 via a partially-electrically-insulated introducer needle 150 and a spacer 159 along the same tissue tract previously traversed and/or produced by the insertion of that biopsy device 120 through the cannula 150. The electrode 120 has an elongated shaft 124, a hub 122 at the shaft proximal end, and a tip point 125 at the shaft distal end. Cannula 150 includes an elongated cylindrical shaft 154 with lumen therethrough, a hub at the shaft proximal end, and a opening 154C at the shaft distal end, the hub 152 having a proximal female luer opening. The shaft 154 includes an electrically-insulated proximal portion 154A, and an electrically-conductive distal portion 154B, which can be referred to an “active tip”. Spacer 159 includes distal male luer that interlocks with the female luer of the introducer hub 152 (this internal interlocking being shown as dotted/dashed lines in FIG. 1A). Electrode 120 includes cable 121 to the generator 100, hub 122, coupling feature 123 (e.g. a male luer that couples with the spacer 159 or the cannula hub 152, this internal coupling being shown as dotted/dashed lines in FIG. 1A), electrically-conductive shaft portion 124 (which is shown as a dotted line 124A within the lumen of the cannula shaft 154) and electrically-conductive distal end 125, which is the distal end of biopsy-collection shaft within shaft portion 124. The cooperation of shafts 124 and 125 are used to captures s tissue sample, as is known to those skilled in the art of tissue biopsy. The shafts 154 and 124 are cylindrical. The insulated portion 154A of cannula shaft 154 is cylindrical and electrically non-conductive, and it prevents or substantially limits the flow of RF signal output from the generator 100 to tissue 160. Electrical output from generator 100 is conducted to the electrode shaft 124 and 125 within the electrode 120, and to the active tip 154B of the cannula by coaxial contact between the electrode shaft 124 and the inner lumen of the cannula shaft 154 and its uninsulated distal end 154B. Together, the cannula distal end 154B and protruding portions of the biopsy shaft 124 and 125 form a combined electrically-conductive active tip from which the output of generator 100 flows from its first output pole 104 to the tissue 160 and then to the ground pad 140 which is connected to the other output pole 105 of the generator 100. Ground pad 140 is applied to the skin surface of body 160 and carries return current from the combined active tip 154B/124/125 to the generator 100 via cable 141. Biopsy electrode 120 includes connector 129 to which cable 121 is attached removably (in other embodiments, the cable 121 can be inseparably attached to the electrode 120). The RF output through tip 154B/124/125 produces tissue heating around the tip 125, which heating is shown as the coagulation zone 161A, which is axially symmetric around the electrode and cannula shafts. The electrode cable 121 connects to a first output jack 104 of the generator. The ground pad cable 141 connects to a second output jack 105 of the generator. The generator produces an RF potential across pins of the jacks 104 and 105. The generator 100 includes a Start/Stop toggle buttons 101A and 101B for starting and stopping the RF output to connected electrodes and ground pads. Button 101A starts an output mode specialized for ablation of the biopsy needle tract (also known as a “tract burn” or “track burn”). Button 101B starts and stops an output mode for generating a large ablation around the combined active tip of the electrode and cannula, and in some embodiments, can provide further user prompt that allows for selection among non-cooled, cooled, perfusion, or other ablation modes. The generator 100 includes user-adjustable settings 102. In some embodiments, the settings 102 can include some or all of the following: a target output level for tract ablation mode configured to induce boiling around the combined active tip of the electrode and cannula, a target output level for ablation mode configured to maximize the size of ablated tissue around the combined active tip, a target temperature for ablation mode, a maximum output-on time for ablation mode, a high limit on the electrode impedance that triggers automatic turn-off of the generator output, selection of control mode, selection of non-cooled or cooled ablation, selection of ablation or tract burn modes, and selection of manual or automatic control. In some embodiments, the generator settings can include target voltage, current, and/or power settings. The generator includes a knob 103 for the option of manual control of the generator output level. The generator display 106 includes numeric display of electrode and generator measurements, including the electrode temperature 113 (that is measured using a temperature sensor included in the electrode tip 125), the impedance between the electrode 120 and the ground pad 140, the generator output level 114 (which in this embodiments is RF current, and can be voltage, power, and/or current in other embodiments), and the elapsed output-on time 111. In this embodiment and mode of the generator display 106, the temperature 113, impedance 112, and current 114 are each graphed on vertical axis 110 as a function of the axis 111A for the time 111; the respective graphs being 113A for temperature 113 (dashed line with endpoint labeled “T”), 112A for impedance 112 (solid line with endpoint labeled “Z”), and 114A for current 114 (dotted line with endpoint labeled “I”). The user can monitor these measurement displays as output is delivered from the generator to produce ablation zone 161A.

Also shown is stylet 180 that has an elongated cylindrical shaft 184, hub 182 at the shaft proximal end, and point 185 at the shaft distal end. Stylet 180 can be used to obdurate the introducer cannula 150 during its initial insertion into the tissue 160. After such insertion, the stylet 180 is replaced by the biopsy device, which is inserted one or more times to collect tissue samples. After such collection, the ablation 161A is generated. In FIG. 1A, example graphs are shown that depict generator output that is configured to cause boiling in the tissue.

An advantage of the system in FIG. 1 is that, without having to move the introducer cannula 105 or the biopsy electrode 120 during ablation, the ablation zone 161A encompasses all portions of the cannula 150 and biopsy device 120 to which tumor cells might adhere and be later displaced to health tissue regions by withdrawal of the electrode 120 and cannula 150 from the body 160, and all regions of the tissue in which tumor cells might have been displaced by the biopsy process. After such ablation the cannula 150 and biopsy electrode 120 can be withdrawn without risk of dragging tumor cells into more superficial part of the tissue 160 thereby avoiding spread of cancer. When the biopsy device 120 is withdrawn into the introducer 150, tumor cells can be dragged to the distal opening 154C of the cannula 150. In some cases, the distal end 154B of the cannula 150 may be positioned within the tumor 169, as show in FIGS. 1A and 1B. An advantage of the system in FIG. 1 is that the ablation 161A encompasses the distal end of the cannula 154B and kills any tumor cells at that location before the cannula 150 is withdrawn from the tissue.

The cannula shaft 154 can be constructed from an ultra-thin-wall stainless steel hypodermic tubing, over which electrical 154A insulation is applied. The electrode outer shaft 124 can be composed from stainless steel tubing. The inner shaft 125 can be composed from stainless steel. The shaft 124 outer diameter can be configured to have tight clearance with the inner lumen of the cannula shaft 154 for smooth insertion, e.g. 0.001″ to 0.0015″. The stylet shaft 184 outer diameter can have similar tight tolerance to the cannula shaft inner lumen. Cannula shaft 154 can be composed from, for example, be 21UTW, 19UTW, 17UTW, 15UTW, or 13.5UTW hypotube. The electrode outer shaft 124 can be composed from, for example, 22, 20, 18, 16, or 14 gauge stainless steel tubing. In some embodiments, the cannula shaft tubing inner diameter entailed by using 21UTW, 19UTW, 17UTW, 15UTW, and 13.5UTW tubing can be paired with the electrode outer shaft 124 outer diameter 22, 20, 18, 16, and 14, respectively. In some embodiments, the nominal clearance between the inner diameter of the shaft of the cannula 150 and outer diameter of the electrode 120 shaft can be in the range 0 to 0.003″. The electrical insulation 154A can be composed from PET, PTFE, polyimide, or another electrically-insulative material or tubing, and can have wall thickness 0.0005″, 0.001″, 0.0015, 0.002″, or another wall thickness, for example a wall thickness in the range greater than 0″ and less than 0.0025″ or more, or for example, a wall thickness that is configured to limit leakage current through the insulation and to minimize transitional thicknesses between the outer diameter of the insulated region 154A and the conductive region 154B of the cannula 150.

The length of the conductive region 154B (“Cannula active tip”) of the cannula 150 can be configured to produce different ablation sizes or to allow different tolerances for insertion of the cannula shaft 154 into a suspected tumor. For example, the cannula active tip can have length 0.1, 0.5, 1, 1.5, 2, 2.5, 3, 4, 5 cm or more, and a length in the range 0.1 cm to 5 cm or more, in the direction of the cannula shaft 154 length. The length of the conductive region 124/125 (“electrode active tip”) of the electrode 120 that protrudes from the cannula distal open 154C can be configured to produce different ablation sizes or to allow for collection of tissue samples at different distances distal to the cannula distal end 154C. For example, the electrode active tip can have lengths 0.1, 0.5, 1, 1.5, 2, 2.5, 3, 4, 5 cm in the direction of the cannula shaft 154 length. In some embodiments, the cannula active tip and electrode active tips can have the same lengths. In some embodiments, the cannula active tip and electrode active tips can have the different lengths. In some embodiments, the cannula active tip and electrode active tips can have 2.5 cm length, which has special advantage in that is corresponds to the typical protrusion of a biopsy device from a coaxial introducer and provides sufficient margin of insertion of the cannula into a suspected tumor region.

The distal end 154C of the cannula can be square cut and blunt as shown in FIG. 1A. In other embodiments the distal end of the cannula can include a bevel and/or sharpened edges to facilitate cutting. As shown in FIG. 1A, the stylet 180 has a sharp trocar tip. In other embodiments, the stylet 180 can have a tip that shaped to match the bevel and/or or sharp edges of the cannula distal end 154C to facilitate penetration. In some embodiments, the distal end of electrical insulation 154A on the cannula shaft can be smoothed to provide a smooth transition to the cannula active tip portion 154B; this has the advantage of providing smooth insertion. In some embodiments, the cannula can include a radiopaque marker and/or an echogenic marker at the proximal end of the cannula active tip 154B, so the proximal end of the combined active tip of the electrode 120 and cannula 150 can be viewed in x-ray, CT, or ultrasound relative to anatomy, such as the edge of the suspected tumor region 169. In other embodiments, the radiopaque marker and/or echogenic marker can be under the distal end of the electrically-insulated cannula shaft portion 154A, thereby providing the same advantage.

FIG. 1A shows an exemplary method of the present invention wherein the tissue tract for a biopsy device 120 (formed by the cannula 150 and the biopsy device 120) is coagulated after biopsy is performed. FIG. 1A shows a combination biopsy and ablation electrode 120 introduced into a body 160 using a cannula 150 having a proximally-insulated shaft 154 partially-insulated cannula 150 and spacer 159 after collection of a tissue sample from target tissue 169 by prior use of the biopsy device 120 via the cannula 150 and spacer 159. Generator 100 delivers high-frequency electrical output to the tissue 160 via the combined electrically-conductive distal ends (“154B, 124 and 125; “combined active tip”; “active tip of the assembly”) of the biopsy electrode 120 and the cannula 150 such that tissue around the combined active tip is heated to a destructive temperature forming ablation zone 161A, thereby coagulating any bleeding that was caused by insertion of the biopsy device 120 into the tissue and/or thereby killing any tumor cells from within region 169 that might adhere to the cannula distal end 154B or to the biopsy device 120 and that might otherwise be dragged into heathy regions of the tissue 160 by withdrawal of the cannula 150 and/or biopsy device 120. The generator 100 show an exemplary operational mode wherein high generator output (e.g. current) is used to induce rapid boiling and coagulation around the entirety of the combined active tip.

FIG. 1B shows an exemplary method of the present invention wherein a biopsy is performed immediately before ablation of a tumor 169. One advantage of the system and method shown in FIG. 1B is that tissue samples can be easily collected before intended ablation to support later tissue analysis, to provide more detailed diagnosis, to guide more specifically post-ablation management of a cancer disease process that may be more widely spread in the body, to guide additional post-ablation treatment of the tumor (e.g. by radiation therapy or chemotherapy or immunotherapy), or to provide data for further research into cancer.

FIG. 1B shows an exemplary method of the present invention using the system of FIG. 1A wherein a (suspected or confirmed) tumor 169 is ablated using the combination of biopsy electrode 120 and cannula 150, after biopsy was performed using biopsy device 120. FIG. 1B shows a dual-function biopsy and ablation electrode 120 introduced into a body 160 using a cannula 150 having a proximally-insulated shaft 154, without using spacer 159, after collection of a tissue sample from target tissue 169 by prior use of the biopsy device 120 via the cannula 150 (either with or without use of the spacer 159). Electrode 120 is further connected to a pump 130 that circulates fluid (such as sterile water or saline) within the electrode shaft 124 via tubing 133 connected to electrode connector 129; the pump having start/stop control 131, settings control 132, and link 134 to the generator 100 for common control and/or monitoring and/or coordinated operation. The tubing 133 can include inflow and outflow tubing that are with combined in a single dual-lumen assembly, or that are physically separate. Generator 100 delivers high-frequency electrical output to the tissue 160 via the combined electrically-conductive distal ends (154B, 124 and 125; “combined active tip”; “active tip of the assembly”) of the biopsy electrode 120 and the cannula 150 such that tissue around the combined active tip is heated to a destructive temperature forming ablation zone 161B, thereby coagulating any bleeding that was caused by insertion of the biopsy device 120 into the tissue, and thereby killing (or attempting to kill) the entire tumor 169 and any tumor cells from within region 169 that might adhere to the cannula distal end 154B or to the biopsy device 120 and that might otherwise be dragged into heathy regions of the tissue 160 by prior withdrawal of the cannula 150 or biopsy device 120. The generator 100 show an exemplary operational mode wherein high generator output (e.g. current) is used to induce intermittent boiling of the tissue in response to impedance increases and/or drops in the output current or power. It is understood that ablation can be performed with or without cooling and that other modes of ablation can be performed including wherein fluid (such as hypertonic saline) is perfused through the electrode 120 to the tissue, wherein temperature control is used, wherein power or voltage control is used, or wherein other methods of ablation control are used as are known to one skilled in the art of tissue coagulation, tissue ablation, and tract burns. It is understood that both tract burn (e.g. FIG. 1A) and tumor/target ablation (e.g. FIG. 1B) can be performed with or without the spacer 159. Note that, in another method of the present invention, biopsy can also be performed after ablation to assess the effect of ablation on tissue.

In one example, FIG. 1B shows a tumor ablation performed using the electrode 120 and cannula 150 without a spacer 159 wherein the distal end of the electrode is positioned near the edge of the tumor 169, after the electrode biopsy device 120 was used to collect a tissue sample using the cannula 150 and the spacer 159 from a location near the center of the tumor 169 as shown in FIG. 1A (and with or without the tract burn 161A). This demonstrates one advantage of the system of FIG. 1 wherein, without moving the introducer cannula 150, tissue samples can be collected from a location appropriate for biopsy of a tumor 169 and the active tip of a combination ablation device (i.e. electrode 120 and 150) can be positioned in a tumor 169 appropriate for complete ablation of that tissue.

FIG. 1C shows separate components of the system separately. Some or all of these components can comprise a disposable biopsy kit for use with a generator 100, sold separately. Shown are the biopsy electrode 120, ground pad 140, spacer 159 (that allow for biopsy at more superficial locations through the cannula 150), the stylet 180 with trocar end that facilitates insertion of the cannula 150 into tissue, and the cannula 150. Lines 158 show how each of the electrode 120, spacer 159, stylet 180 can each be inserted into the lumen of cannula 150.

FIG. 1D shows another view of the items in FIG. 1C, wherein one embodiment of the biopsy electrode 120 is shown with the inner biopsy stylet 125S removed. The biopsy stylet 125S includes a hub 125H at the proximal end of the shaft 125A, which has distal end 125. Dotted line 158D shows show the biopsy stylet 125S is inserted into the remainder of biopsy electrode 120 and through its outer shaft 124. One advantage of this embodiment is that a tissue sample can be collected by withdrawal of biopsy stylet 125S without moving the biopsy outer shaft 124, which makes up the predominance of the electrode active tip. This further increases the likelihood of complete and accurate ablation of the biopsy tract. In other embodiments of biopsy electrode 120, the stylet 125S is not removable by the user, and tissue samples are collected with withdrawal of the entire biopsy electrode 120 from the tissue 160.

In other embodiments, the electrode 120 can conduct generator output to the cannula active tip 154B by other mechanisms, such as conductive contacts within the electrode hub 122 and cannula hub 152.

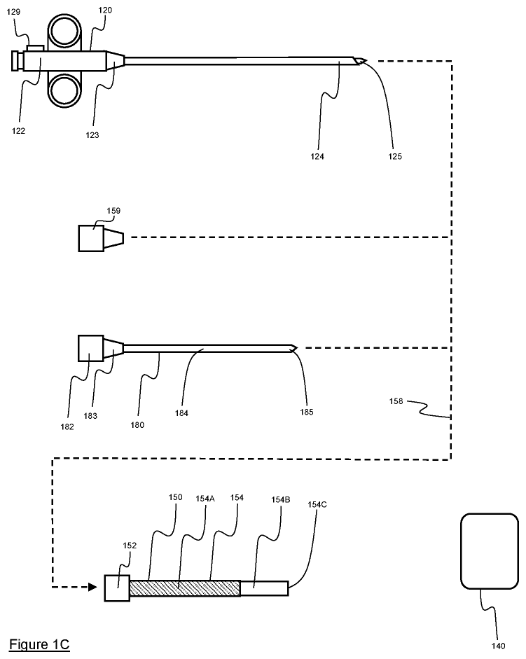

FIG. 2 refers collectively to FIGS. 2A and 2B.

FIG. 2A shows another embodiment wherein the monopolar biopsy electrode 120 of FIG. 1 is replaced by a bipolar biopsy electrode 220, which includes proximal electrically-conductive shaft 224 that is within the lumen of cannula 154 (and thus shown as dotted lines), intermediately electrically-insulated region 225A that is partially within the shaft lumen of cannula 150 (those parts within the cannula being shown as dotted/interrupted region 225AA) and partially protruding from that lumen, and distal electrically-conductive regions 225B and 225C that form the electrode active tip and the core biopsy mechanism. The first output pole of the generator 100 is connected to the distal electrode tip 225B and 225C, and the second output pole of the generator 100 is connected to the proximal electrode shaft 224. The electrode tip 225B and 225C is electrically insulated form electrode shaft 224 within the electrode. Electrode shaft 224 conducts the generator output from the second generator pole to the cannula active tip 154B by conductive contact between the shaft 224 and the lumen of cannula 154 when the electrode 220 is inserted into cannula 150. As such, generator output flows from the electrode tip 225B and 225C to cannula tip 154B, thereby heating tissue in between to form ablation zone 261, without the use of a ground pad. In other embodiments, the electrode 220 conduct the second generator output pole to the cannula tip 154B via a different means of electrically-conductive coupling, such as mating electrical contacts included in the cannula hub 152 and electrode hub 223.

FIG. 2A shows another embodiment of the present invention in which the monopolar electrode 120 of FIG. 1 has been replaced by a bipolar electrode 220, and in which the generator 100 presents user controls 201 and 202, rather than 101A, 101B, 102. Button 201 provides for starting and stopping generator output and, in some embodiments, output mode selection. Controls 202 provide for certain control settings, including electrode target temperature, ablation time, target electrode current level, the impedance level above which output is automatically discontinued by the generator, and a control mode setting. In the example mode shown in FIG. 2A, the generator regulates its output level to control the measured temperature of the electrode 220. The bipolar electrode includes generator cable 221, hub 220, male luer 223, proximal conductive shaft 224, insulated shaft portion 225A, and distal electrode contact 225B and 225C. The electrode shaft comprises 224, 225A, 224B, and 225B and is substantially cylindrical. Electrode 220 is a bipolar electrode and a core biopsy device wherein generator RF output flows from electrode active tip 225B and 225C through the tissue 160 to cannula active tip 154B (which is electrified by electrode shaft 224 within it). A ground pad 140 is omitted relative to FIG. 1. The said flow of RF current generated tissue heating around and between the electrode and cannula tips thereby producing ablation zone 261, which can be substantially axially symmetric. In the embodiment shown in FIG. 2A, the display 106 of generator 100 replaces the line graphs of FIGS. 1A and 1B with dynamic graphs. The first graph 213 plots the temperature reading 113 as line 213A and plots the maximum measured temperature as line 213B. Line 213A can be a bar graph in other embodiments. The second graph 212 plots the impedance reading 112 as line 212A and plots the minimum measured impedance as line 212B. Line 212A can be a bar graph in other embodiments. In other embodiments, other readings can be plot using dynamic bar graphs or other similar graphs, and other maximum, minimum, and/or average values can be plotted in additional to the real-time readings. In some embodiments, minimums and maximums of impedances, temperatures, and output levels can be displayed numerically and in other graphical forms.

FIG. 2B shows separately the probe components of the system of FIG. 2B. Lines 258 show how each of the electrode 220, spacer 159, stylet 180 can be inserted into the lumen of cannula 150. The outer shaft 224 can have outer diameter configured to have tight clearance relative to the inner diameter of the cannula shaft 154, e.g. between 0 and 0.003″ clearance. The electrode outer shaft 224 can be composed from, for example, 22, 20, 18, 16, or 14 gauge stainless steel tubing, for example, corresponding to the shaft tubing of the cannula composed of 21UTW, 19UTW, 17UTW, 15UTW, or 13.5UTW hypotube, respectively. In some embodiments, the insulated region 225A of the electrode 220 can have a same outer diameter as the outer shaft 224. In some embodiments, the distal tip region 225B of the electrode 220 comprises an stainless steel hypotube that is within, and electrically insulated by electrical insulation layer 225A from, the axial lumen of the outer shaft 224; this these embodiments, the electrical insulation 225A can be composed from PET, PTFE, polyimide, or another electrically-insulative material or tubing, and can have wall thickness 0.0005″, 0.001″, 0.0015, 0.002″, or another wall thickness, for example a wall thickness in the range greater than 0″ and less than 0.0025″ or more, or for example, a wall thickness that is configured to reduce capacitive coupling between the shaft 225B and shaft 224 and to minimize transitional thicknesses between the shafts 225B and 224. The length of the cannula active tip 154B and the length of the electrode active tip 225B and 225C can be configured to create a confluent ablation zone 261 around the combined active tip region 154B, 225A, 225B, and 225C of the combination of the cannula 150 and the electrode 220. The combined active tip region can have length 0.5, 1, 1.5, 2, 2.5, 3, 3.5, 4, 4.5, 5 cm or more, or a length in the range 0.1-5 cm or greater. The length ratio of the cannula active tip 154B, inter-tip region 225A that protrudes from the cannula, and the electrode active tip 225B and 225C can be configured to create a confluent ablation zone 261 around the combined active tip region 154B, 225A, 225B, and 225C of the combination of the cannula 150 and the electrode 220. For example, the said ratio can be 2:1:2, respectively. For example, the said ratio can be 1:1:1, respectively. For example, the said ratio can be produced by lengths 2.5 mm:1 mm:2.5 mm, 4 mm:2 mm:4 mm, 6 mm:3 mm:6 mm, 8 mm:4 mm:8 mm, or 10 mm:10 mm:10 mm. In some embodiments, the distal tip 225B and 225C can be shorter in length than the proximal tip 154B so that current density and tissue heating is emphasized around the distal tip. In some embodiments, the distal tip 225B and 225C can be longer in length than the proximal tip 154B so that current density and tissue heating is emphasized around the proximal tip.

It is an advantage of the present invention that the electrode and biopsy function are combined in a single device, either 120 or 220 because among other advantages, (1) it increases the likelihood that the same biopsy device will repeatedly follow the same tissue tract thereby increasing the likelihood of destroying tumor cells and coagulating blood vessel along the biopsy tract; (2) it simplifies the procedure steps for the physician; (3) it reduces the number of pieces of equipment used for biopsy and ablation; (4) it increases the likelihood that a physician will take the time to reduce the chance of cancer spread; and (5) it reduces the total cost of equipment for biopsy and ablation.

In some embodiments of biopsy devices 120 and 220, the outer shaft 124, 224 is left in place in the tissue 160 while the tissue sample is collected using shaft connected to tip 125, 225, and then the combination of the cannula 150 and the electrode 120 are used for ablation or tract burn via the combined active tip of the cannula 150 and the biopsy device 120, 220, respectively. An example of such a removable biopsy stylet 125S is shown in FIG. 1D. This has the advantage that it further increases the chance that the biopsy tissue tract is fully coagulated.

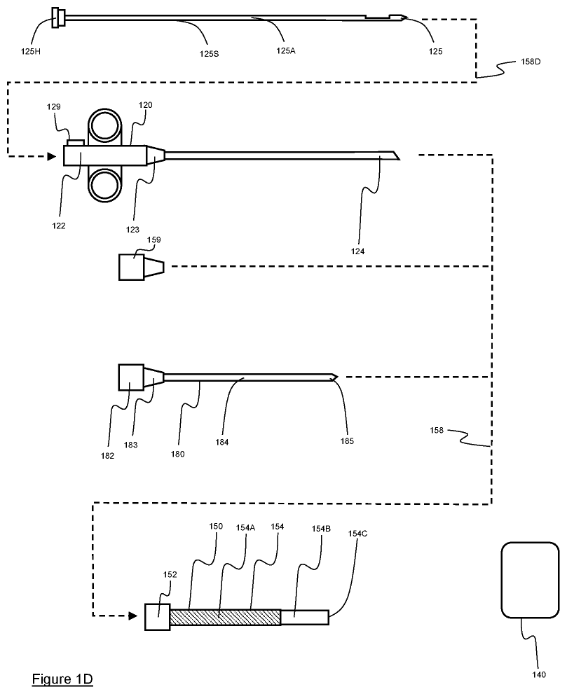

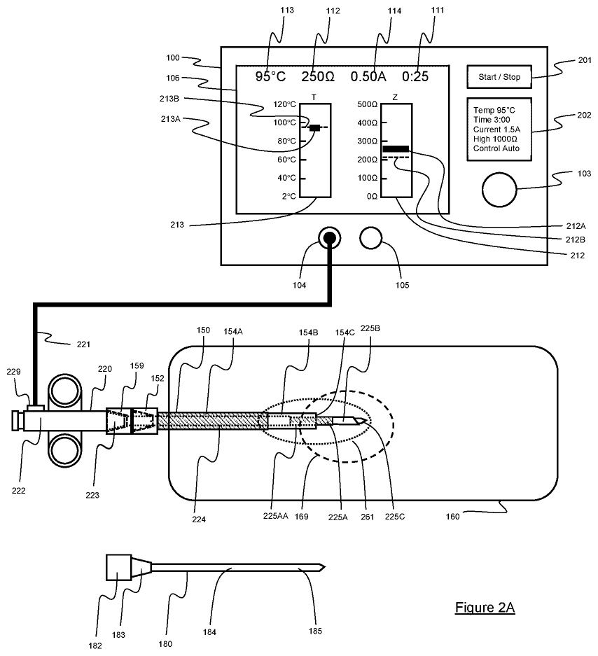

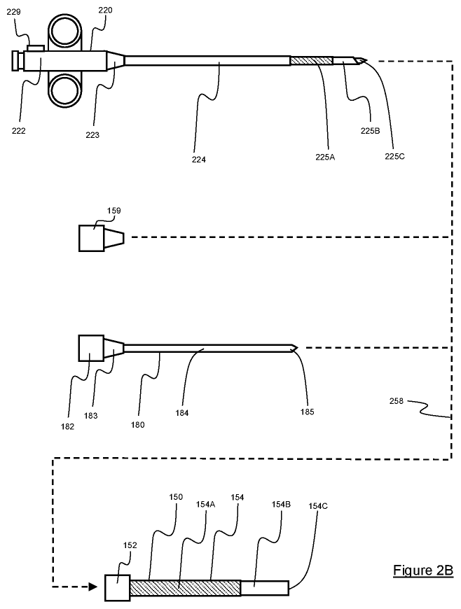

In some embodiments, the system of FIG. 1 can be used with the methods and generator features of FIG. 2 and FIG. 4 and FIG. 5. In some embodiments, the system of FIG. 2 can be used with the methods and generator features of FIG. 1 and FIG. 4. In some embodiments, the system of FIG. 3 can be used with the methods and generator features of FIG. 1, FIG. 2, FIG. 4, and FIG. 5.

In other embodiments, the line graphs of FIG. 1 and of FIG. 3 can be used for a bipolar electrode, such as electrode 220 or 520. In other embodiments, the graphs of FIG. 2 can be used for a monopolar electrode, such as electrode 120. Monopolar tract ablation has the advantage of producing a more extended ablation zone along the active tip, but requires the use of a ground pad. Bipolar tract ablation has the advantage not requiring a ground pad, but produces a more focused and smaller ablation between the active tips that can require more controlled electrode withdrawal. In other embodiments, a generator mode configured to produce boiling around the tip of a bipolar electrode 220 can be used. A control mode that induces boiling around the active tip of a monopolar electrode 120 or a bipolar electrode 220 has particular advantage for longer monopolar active tip lengths (e.g. the length spanned by 154B, 124, and 125 in excess of 2 cm, for example, 2-6 cm or longer), or bipolar active tips (e.g. proximal tip exposure 154B and the distal tip exposure 225B and 225C) because it helps ensure ablation occurs around the entirety of the active tip(s) (and the insulative region between the bipolar tips) because regions of the active tip(s) with lower current density initially, later deliver higher current density after tissue boiling occurs around, and impedes current flow from, the initially-higher-current-density regions of the active tip(s). For example, current density is initially higher at the distal and proximal ends of a long monopolar active tip, leading to a more thin, lower temperature ablation around the longitudinal middle of the active tip when current density is initially lower; however, once tissue starts to boil and hinder current flow from the distal and proximal ends of the active tip, current density increases in the longitudinally middle portion of the active tip, thereby increasing the rate of heating in tissue around the longitudinally middle portion of the active tip. Similarly, for bipolar active tips, current density is initially higher at the points of the active tips that are closer to each other (because current follows the path of least resistance), for example, at the distal part of the proximal active tip 154B and at the proximal part of the distal active tip 225B. This leads to less tissue heating and ablation at the more proximal aspects of the proximal active tip 154B and at the more distal aspects of the distal active tip 225B and 225C. However, after boiling in tissue near the initially high-current aspects of the bipolar active tips, current density increases and produces more tissue heating at the initially low-current aspect of the bipolar active tips. In contrast, if the generator is configured to control temperature measured at the distal end of the electrode (125 or 225C), then the more proximal parts of the combined active tip (e.g. the proximal part of 154B) may be cooler and not create the same extent of ablation nearby.

FIG. 3 shows an alternative embodiment of the monopolar system of FIG. 1C wherein the electrode 320 is a separate device from the biopsy device 370. The electrode 320 includes generator connector 321A, cable 321, hub 322 including cannula-mating hub portion 323, shaft 324 whose proximal end is connected to hub 322 and which has blunt distal end 325. In some embodiments, connector 321A can include inflow and outflow fittings and/or connectors for inflow and outflow tubing included in cable 321 that provides for inflow and outflow for fluid to the electrode shaft 324 for internally-cooled or perfusion ablation operation of the electrode 320 and cannula 150. In the system shown in FIG. 3, biopsy device 370 does not have a generator connection and is a conventional biopsy device. Lines 358 show how the core biopsy device 370, electrode 320, spacer 159, and stylet 180 can each be individually inserted into the lumen of the cannula 150, one at a time. The biopsy device 370, electrode 320, spacer 159, stylet 180, and cannula 150 are drawn schematically with specific length relationships and aligned longitudinally with the cannula to depict their longitudinal position relative to the cannula when fully inserted so that their hubs abut the cannula hub 152. The electrode 320 and biopsy needle 370 are configured to have substantially the same shaft length and degree of protrusion from the cannula 150 (and from the combination of the cannula 150 and the spacer 159); this has the advantage that electrode ablates the full extent of the biopsy device tract in the tissue after the biopsy device is removed from the tissue. In another embodiment, the electrode 320 can protrude farther from the cannula 150 distal opening 154C (for example 1-5 mm or up to 10 mm) than does the biopsy device 170 to ensure full ablation of the biopsy device's tissue tract, any bleeding, and any tumor cells displaced distally (for example, having been pushed distally by insertion of the biopsy device). The electrode distal end 325 is blunt; this has the advantage that it will tend to follow the tissue tract previously cut by the biopsy device 370 after the electrode 320 replaces the biopsy device 370 (as opposed to a sharp distal end that might cut a different tract through the tissue). In other embodiments, the distal tip of the electrode 325 can be sharp to provide for tissue penetration more distal to the distal end of the cannula 150. In other embodiments, the distal tip of the electrode 325 can include a protruding temperature sensor that provides for tissue temperature measurement more distal to the distal end of the electrode 320, for example, for temperature control if the electrode shaft is cooled. One advantage of the system in FIG. 3 is that a disposable electrode kit can include an electrode 320, cannula 150, stylet 150, and optionally a spacer 159 configured for use for a widely-used biopsy device 370, such as one manufactured by Merit, Carefusion (branded Temno), Inrad, Apriomed, and Bard. Multiple kits could be marketed, each of which could be specialized to the biopsy device of a particular manufacturer to combine the advantage of multiple particular biopsy devices with those of the system of the present invention. This could have a substantial advantage in terms of improving the safety of biopsy, independent of the particular device and biopsy method used. Another advantage of the system in FIG. 3 is that more complex electrode features can be more easily, effectively, and/or cost-effectively integrated into a separate electrode 320 rather than integrated all featured into a combination biopsy and electrode device, such as 120 and 220. For example, in some embodiments, a separate electrode 320 can more easily and cost effectively incorporate a temperature sensor, for example, at or near the distal end 325 of the electrode 320. For example, in some embodiments, a separate electrode 320 can more easily and cost-effectively incorporate internal cooling of the electrode 320 to enlarge ablation size, wherein the fluid is circulated through the electrode shaft 324 via pump 130 connected to the electrode via an inflow and outflow tube, and that fluid circulation can be more effective at cooling the electrode shaft 324 because it can utilize a larger cross-sectional portion of the electrode shaft 324 because the same shaft doesn't also need to provide for biopsy functions.

It is an advantage of the present invention that the introducer 150 and electrode 320 can be configured to be compatible with popular biopsy devices, which in some embodiments device 370 can represent, said compatibility being characterized, at least in part, by the inner diameter of the introducer 150 providing tight tolerance to the outer diameters of both the electrode 320 and the compatible biopsy tool 370, and/or by the electrode 320 and biopsy device 370 having substantially similar lengths, and/or the introducer being configured to have ultra thin wall thickness of electrically-conductive and electrically-insulative portions to facilitate tissue insertion and the likelihood of bleeds. A tight tolerance from the outer diameters of the biopsy device 370 and electrode 320 to the inner diameter of the introducer 150 (e.g. 0.0005″ to 0.0015″) is advantageous to help ensure that the electrode 320 will follow the same tract in the tissue 160 as did the biopsy device 370, and thereby to ensure that tissue touched/cut by the biopsy device 170 is reliably coagulated by the electrode.

In another embodiment, the electrode 320 can be a bipolar electrode with shaft and generator connection features similar to that of the electrode 220, i.e. a proximal shaft insulated from a distal electrode shaft portion, connected to a second pole of the generator output, and configured to conduct the potential of that pole to the electrically-conductive distal end of the cannula 154B when assembled with the cannula; an intermediate electrically-insulated shaft portion; and the distal electrode shaft portion connected to a first pole of the generator output.

In some embodiments, it can be advantage for the electrode 370 to be slightly longer (e.g. 1-5 mm or more) than then biopsy device 370, to ensure ablation all the way to the distal end of the tract formed by the biopsy device. In some embodiments, it can be advantage for the electrode 370 to be the same length as the biopsy device 370, to ensure that the blunt-tip electrode does not push tissue distal to the tract formed by the biopsy device and thereby potentially displace anatomy, tumor cells, or cause tissue bleeding.

In some embodiments, the system of FIG. 3 can be used in the methods and generator features of FIGS. 1A, 1B, 2, 4, 5A, and 5B.

Electrodes 120, 220, 320, 520, and cannulas 150 and 550 can be constructed in a number of ways in accordance with the present invention. The shaft of each probe 120, 220, 320, 520, 150, 550 can be constructed from stainless steel hypodermic tubing, respectively; such tubing can extend the length of the shaft to the hub 122, 222, 322, 152, respectively. Electrically-insulative shaft portions 225A, 154A can be plastic tubing (e.g. polyimide, polyester, Teflon) or coating (e.g. silicon, Teflon) that covers the proximal length of the shaft 225, 154 for electrode 220 and cannula 150, respectively. Outer shaft 224 of electrode 220 can be constructed from stainless steel hypodermic tubing that overlays the insulation layer 225A. The shaft of cannula 150 can be produced by coaxial nesting of metal tubing (e.g. stainless steel hypotube) within plastic tubing (e.g. polyimide, polyester, Teflon). The shaft of bipolar electrode 120 can be produced by coaxial nesting of metal tubing (e.g. stainless steel hypotube) within plastic tubing (e.g. polyimide, polyester, Teflon). In other embodiments, the electrically-conductive and electrically-insulative shaft portions of electrode 220 and cannula 120 can be constructed by applying cylindrical sections of conductive and non-conductive materials over a central shaft. In other embodiment, electrodes 120, 220, 320, and cannula 150 can be constructed in other way as are known to one skilled in the art.

FIG. 4 refers to collectively to FIGS. 4A, 4B, 4C, 4D, and 4E. FIG. 4 shows a sequence of one embodiments of the use of the system of FIG. 1. In other embodiments, the systems of FIG. 2 and FIG. 3 can be used with the methods of FIG. 4.

First, in FIG. 4A, the cannula 150 has the stylet inserted into its lumen to form a solid tissue-piecing needle. The cannula 150 has a blunt, square cut distal end, and the stylet has a trocar distal end 185 that protrudes from the distal end opening 154C of the cannula 150. The assembled cannula 150 and stylet 180 is inserted into tissue 160, e.g. percutaneously, into a target region 169, e.g. a suspected or confirmed tumor, or another bodily structure for which biopsy is desired. In this example, the distal end of the cannula 154B does not penetrate the tumor 169 as it does in FIGS. 1A, 1B, and 2A.

Then, in FIG. 4B, the stylet 180 has been removed from the cannula 150 and replaced with a core biopsy device 120 whose distal end protrudes beyond the distal end of the cannula 150, for example, by 2.5 cm. The biopsy device 120 is used to collect one or more tissue sample by (possibly repeatedly) inserting and removing the biopsy device 120 from the cannula 150. If the biopsy device is inserted multiple times, the spacer 159 can be optionally used to collect samples at different depths relative to the distal end 154C of the cannula 150. Each time the biopsy device 120 is withdrawn into the cannula 150 to collect sample, tumor cells are potentially dragged to the distal opening 154C of the cannula 150, so that even if the cannula is itself not initially placed in the suspected tumor 169, tumor cells can still be drawn along the biopsy device's tissue tract up to the cannula distal end 154C. As such, there can be a need to ablate tissue at the distal end 154B of the introducer cannula 150. If the cannula 150 were uninsulated, electrifying the cannula 150 could cause heating along the cannula shaft all the way the surface of the tissue 160, potentially causing a skin burn and/or undesired burns to other more superficial tissue.

Then, after tissue collection is complete, the electrode 120 is reinserted into the tissue 160 via the cannula 150 and is connected to the electrode jack of generator 100 via cable 121 at connector 129 as shown in FIG. 4C. The ground pad jack of generator 100 is connected to ground pad 140 via cable 141, wherein ground pad 140 is applied to the skin surface of body 160. In this example, the generator 100 operates in a mode intended to adjust the generator output to control the temperature measured at the electrode distal end 125 to a set value. The graphs of display 106 show the temperature T rising to a set value, the impedance Z dropping to a steady value, and the output level (e.g. current) I reducing to a slowly declining value, for a sufficient duration indicated on the graphs' time axis, each of which complementarily indicate that an ablation zone 461 has formed around the full extent of the combined active tip 154B, 124, and 125, thereby coagulating bleeds and tumor cells along the electrode and cannula tissue tract. At this point, the generator output can be stopped and the biopsy device 120 and cannula 150 withdrawn with reduced risk of bleeding and tumor seeding (i.e. spread of tumor cells due to their being dragged by withdrawal of the electrode and/or cannula). It is a substantial safety and procedural advantage of the system in this application that such coagulation can be performed without moving the insertion cannula 150. It is an advantage that the cannula 150 does not need to be moved because this increases the likelihood that the tract of the biopsy device is actually coagulated, as opposed to some other nearby location being coagulated due to misalignment of the ablation device with the biopsy tract.

In some cases, it may be desired to coagulate a greater extent of the tissue tract, for example, if bleeding is occurring more proximally to the proximal part of the most proximal active tip 154B. In that case, as shown in FIG. 4D, the physician can partially withdraw the cannula 150 and electrode 120 as a unit with the generator output still active to extend the ablation zone from toward the bodily surface, i.e. the ablation zone elongates from 461 in FIG. 4C to zone 462 in FIG. 4D. The user received graphical and numeric and time-dependent feedback about the degree to which the cannula and electrode should be withdrawn and about the extension of the ablation zone in order to create a confluent extended ablation zone without gaps and thin spots by means of the variations in the temperature T, impedance Z, and output level I graphs of display 106, wherein the temperature drops, impedance rises, and output level increases in response to moving the electrode to a cooler, more superficial tissue region, and then the temperature rises, impedance drops, and output level drops indicating that the ablation zone has again formed around the combined active tip.

In some embodiments, the physician can withdraw the electrode 120 from the cannula 150, and/or withdraw the electrode 120 and cannula 150 as a unit, in one or more steps with the generator output still active, creating the extended ablation zone 463 and corresponding graphs on display 106 shown in FIG. 4E. It is an advantage that the biopsy electrode 120 can be withdrawn from the cannula thereby focusing current output from the generator to the cannula active tip 154B to preferentially increase the tissue temperature there. This is an advantage because the more proximal portion 154B of the combined active tip (154B, 124, and 125) can be cooler than the more distal portion 225 of the combined active tip, so focusing heating at the proximal location 154B can ensure full coagulation at the tissue adjacent to the more proximal tip 154B. This is an advantage of insulating the shaft of the cannula (i.e. portion 154B) to improve the safety and reliability of biopsy tissue tract ablation, because in the absence of such insulation, the entire cannula shaft 154 would be electrified, potentially heating tissue along its entire length all the way to the skin surface of the body 160.

It is understood that the generator output can be stopped and restarted intermittently over the sequence shown in FIGS. 4C, 4D, and 4E.

It is understood that the generator operational modes described in FIGS. 1A, 1B, and 2A can be used in the method of FIG. 4.

Note that had the distal end 154B of the cannula penetrated the tumor 169 in FIG. 4A (as it does in FIGS. 1A, 1B, and 2A), if the cannula 150 were withdrawn from the tissue at that point, tumor cells could be dragged along with the cannula causing spread of tumor toward the surface of the tissue 160. An advantage of the system and method shown in FIG. 4A is that even the initial ablation 461 in FIG. 4C encompasses the distal end 154B of the cannula 150 and would kill any tumor cell potentially adhered to the distal end 154B that could be spread to healthy regions of the tissue 160 were the cannula 150 withdrawn without that ablation 461.

Time graphing of temperature and impedance on the same time axis as shown in FIG. 4 has special advantage in that it shows the full time history of these readings, so that the physician user can review them at a glance if the user's attention needs to turn away from the screen at some point during the electrode and/or cannula withdrawal. The shape of these time graphs is also useful itself because it shows the rate of change of these values in addition to the magnitude of the change. For example, a large, fast change in one or both of these values can indicate to user that the electrode and/or cannula has been withdrawn too quickly or too far, and thus that the electrode and/or cannula may need to be re-advanced into the tissue to close a gap in the ablation zone.

Displaying the minimum impedance during a tract burn (as shown by line 212B in FIG. 2, or as shown by the impedance graph 112A in FIG. 1, or the impedance graphs in FIG. 4) is a special advantage of the present invention because impedances are generally interpreted as relative not absolute values. The absolute value of an impedance is confounded by numerous factors unrelated to tissue heating, such as the tissue type around the electrode tip or the distance between a monopolar electrode tip and a ground pad. As such, for tract burn, observation of the change in impedance is relevant to assessing completion of the ablation at the current electrode position. It an advantage for a physician to be able to refer back to the minimum impedance measured as a reference for sufficiently heated tissue for the particular electrode, tissue, and system configuration of a given tract burn. Without such reference, the physician must remember this minimum value, and doing so is difficult when attending to multiple other thing during a dynamic track burn procedure.

The display of temperature of a biopsy tract coagulation electrode per se, and the graphing of the temperature in particular, are each an aspect of the present invention and are each useful because temperature directly measures tissue heating and can be used to control the movement of the electrode and ensure a continuous tract coagulation. The display of impedance of a biopsy tract coagulation electrode per se is useful because it provides the user information about tissue heating around the ablation tip which can be used to control the movement of the electrode and ensure a continuous tract coagulation. The combined display of temperature and impedance of a biopsy tract coagulation electrode provides improved information about heating around the electrode and can be used to further improve movement of the electrode and ensure a continuous tract coagulation.

It is understood that the display features of generator 100 are applicable to tract burns, tumor ablation, tissue ablation, and other sizes and types of coagulation, such as those shown in relation to FIGS. 1, 2, 3, and 4. For example, the graphical display of both the temperature and impedance on the same time axis gives complementary information to the user to help control the withdrawal of an ablation electrode and/or cannula to produce a confluent, uninterrupted tract burn, as shown in FIG. 4. Because the temperature measurement is at a single location, it provides precise information about temperature at one location along the electrode active tip, but does not provide complete information about the heating around the entire electrode tip. On the other hand, Impedance (and impedance changes in particular) provides integrated information about heating along the electrode active tip, but does not provide precise information about tissue temperature. As such, temperature and impedance provide different, complementary information to the user during a tract burn. Displaying these two readings, and in particular graphing them, at the same time along with the generator output level provides rich information that the user can use to avoid gaps in the tract ablation zone.

It is typical to place the temperature sensor of an electrode at the distal end of the shaft. This is advantage because it gives information that the most distal part of the tract is ablated. For a monopolar electrode, the distal tip is the point of maximum temperature; however, for a bipolar electrode, the maximum temperature can be between the tips (i.e. in the insulated region between the tips, e.g. 225A of electrode 220 used with cannula 150). As such, the temperature may go up when a user withdraws a bipolar electrode/cannula during tract burn. This can give a false sense of completeness of the ablation zone because the actual maximum heating is occurring proximal to this location, at the location between the active tips (e.g. 225A). However, the impedance will tend to go down in this situation because the proximal tip has entered cooler tissue. This is a special advantage of displaying both impedance and temperature for a bipolar electrode used for tract burn.

FIG. 5 refers collectively to FIGS. 5A and 5B. FIG. 5 shows another embodiment in accordance with the present invention wherein the dimensions of bipolar electrode 220 and cannula 150 in FIG. 2 are modified to produce the bipolar electrode 520 and cannula 550 in FIG. 5. In particular, the relative lengths of the cannula shaft 554 and proximal electrically-conductive electrode shaft 524 are modified so that when the electrode 520 is inserted fully into the cannula 550, the proximal electrode shaft 524 protrudes from the distal opening 554C of cannula 550, as shown in FIG. 5A by shaft portion 524A. Furthermore, the outer surface area of the cannula active tip 554B is less than the outer surface area of the electrode distal active tip 525B, 525C (Feature 1). Furthermore, the portion of the electrode proximal conductive shaft 524 that protrudes from the cannula distal opening 554C forms a proximal electrode active tip 524A of the electrode (Feature 2). The combined active tip 554B, 524A formed from the cannula active tip 554B and electrode proximal active tip 524A is configured to have a larger surface area than the electrode distal active tip 525B, 525C when the electrode 520 is fully inserted into the cannula 550 (Feature 3). These features individually, in combination, and in total provide special advantages for producing a tract burn by withdrawal of the electrode 520 from the cannula 550 while ablation energy is delivered to the tissue 160 by current flow between the two bipolar active tips, the tips being (1) the distal electrode tip 525B, 525C and (2) the combined active tip 554B, 524A of the cannula 550 and the electrode 520. The advantages include the ability to produce tract burn ablation that includes the distal end of the electrode 525C, the distal end of the cannula 554C, and the length in between, without the use of a ground pad (such as 140). To effect this, with the electrode 520 inserted into the cannula 550 sufficiently far such that the electrode proximal shaft 524 protrudes from the distal end 554C within the tissue 160 (Feature 2), and such that the outer surface area of the combination of the cannula active tip 554B and the protruding electrode outer shaft 524A has greater surface area than that of the electrode distal active tip 525B, 525C (Feature 3), as shown in FIG. 5A. The generator 100 establishes a high-frequency electrical potential difference (such as in the RF range) between the electrode proximal conductive shaft 524 and the electrode distal active tip 525B, 525C via cable 221. The electrode conductive outer shaft 524 contacts the conductive inner lumen of the cannula shaft 554 and thereby brings the cannula active tip 554B to the same electrical potential as is connected to the electrode outer shaft 524. Because of Feature 3, heating is focused at the distal active tip of the electrode shaft 525B and 525C, because it has a smaller surface area, and therefore a generally higher current density, than the proximal combined active tip 554B and 524A. The length of the inter-tip region 525A, when fully protruding from the cannula distal end 554C, is long enough that an ablation forms around the entirety of the distal active tip 525B, 525C, including the distal end of the electrode shaft 525C (Feature 4). As the electrode 520 is withdrawn into the cannula 550 with generator energy still applied, the length of the protruding proximal electrode shaft 524A decreases, and the relative size of, and current density at, the active tips equalizes, thereby forming an ablation at both active tips. As the electrode 520 is further withdrawn into the cannula 550 and, ultimately, the cannula active tip 554B becomes the entirety of the proximal combined active tip because the electrode outer shaft distal end 524A fully withdraws into the cannula lumen, the heating becomes focused at the cannula active tip 554B because the surface area of the cannula active tip 554B is less than the surface area of the electrode distal active tip 525B, 525C (Feature 1). As the electrode 520 is withdrawn into the cannula 550 with generator energy still applied, the protruding length of the inter-tip insulated region 525A further decreases, thereby focusing heating between the top active tips, the distal end of the cannula 554C, and the proximal end of the electrode distal active tip 525B. Importantly, focus of heating at the distal opening of the cannula 554C ablates tumor cells that were dragged to the cannula opening 554C when the biopsy probe 520 was earlier withdrawn to capture a tissue sample. The aforementioned focusing of heating is an advantage because such focus makes the ablation process less sensitive to variations in local tissue characteristics (such as electrical conductivity, thermal conductivity, and heat sinks).

FIG. 5A shows the probe of FIG. 5 in an assembled operational state. FIG. 5B shows the components of the probe system of FIG. 5 separately.

In some embodiments, the spacer 159 can be omitted from the system of FIG. 5. In some embodiments, the hybrid biopsy/electrode 520 device can be configured as a bipolar electrode having the features of the hybrid device 520 except for the biopsy features, and a physically separate biopsy device, such as shown in FIG. 3.

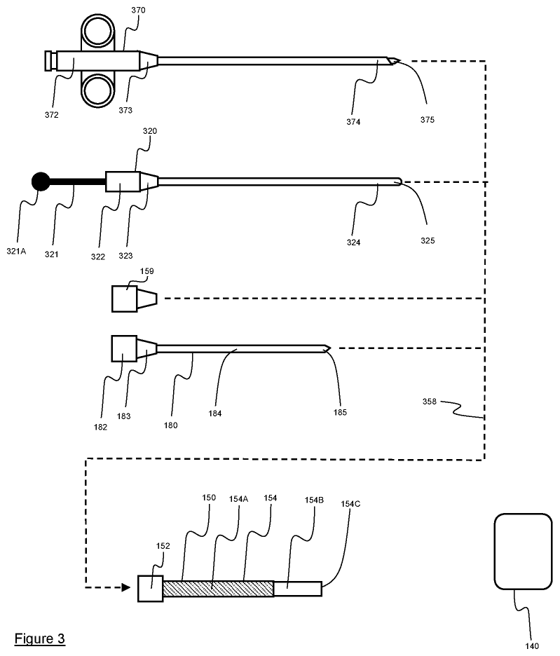

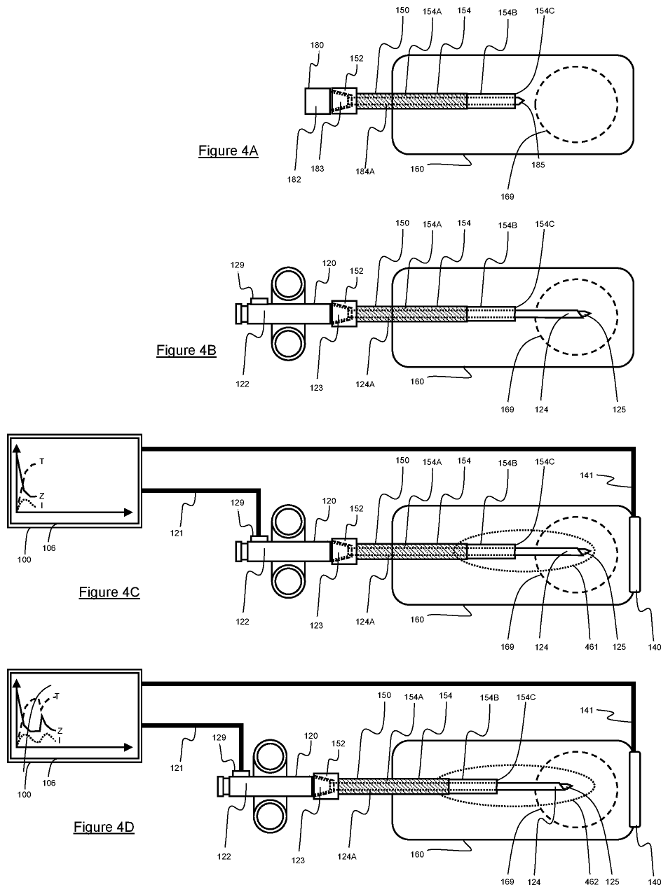

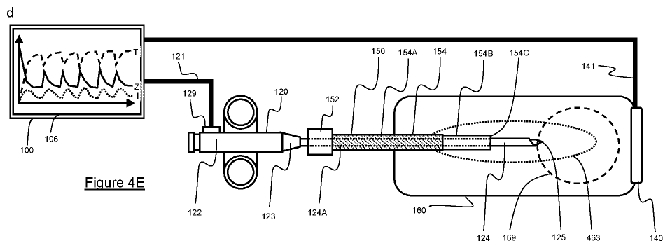

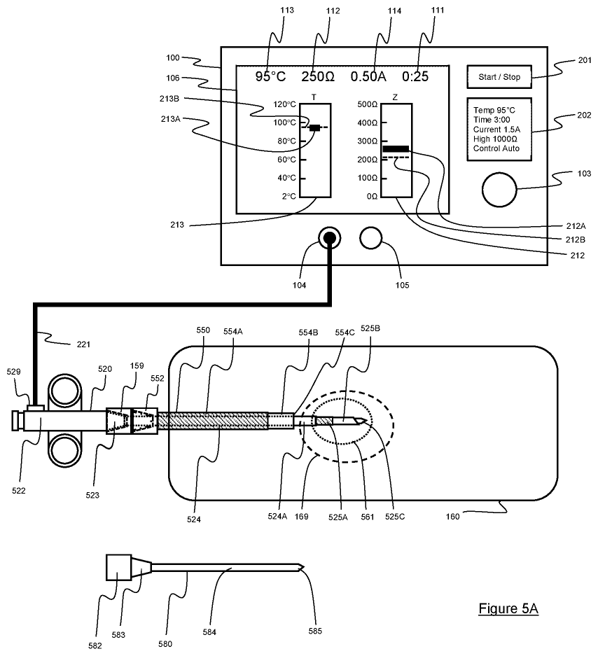

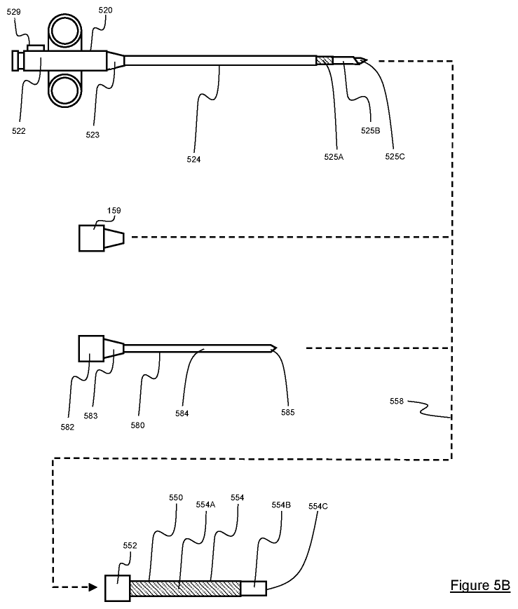

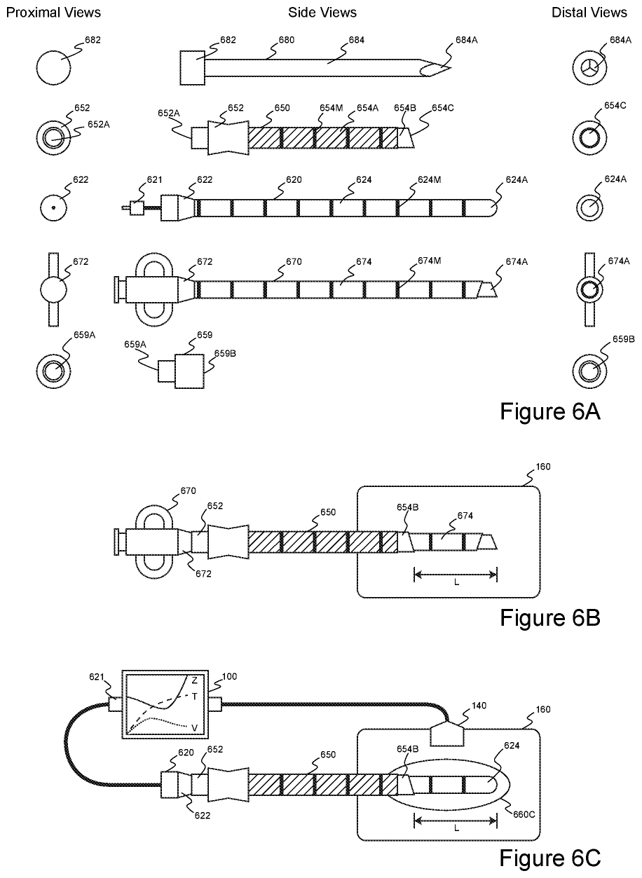

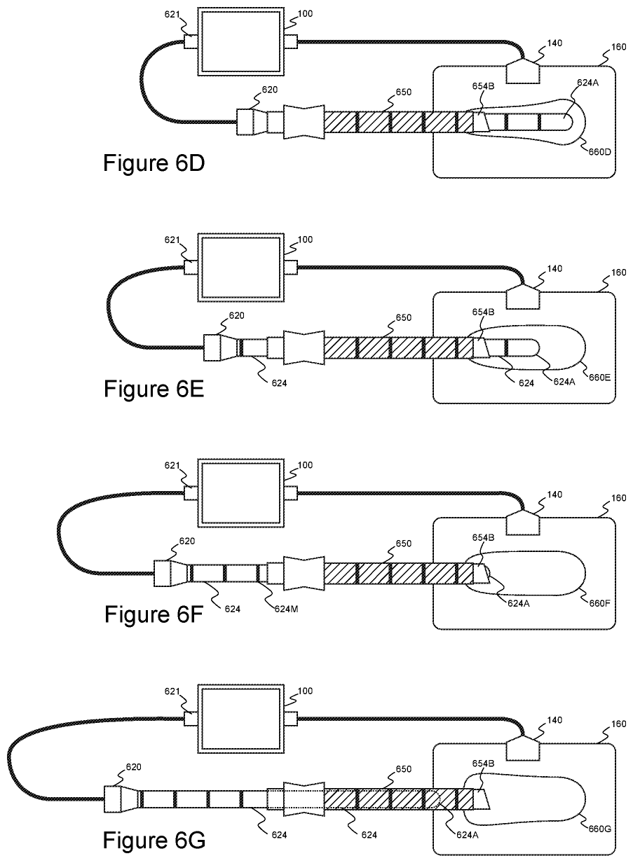

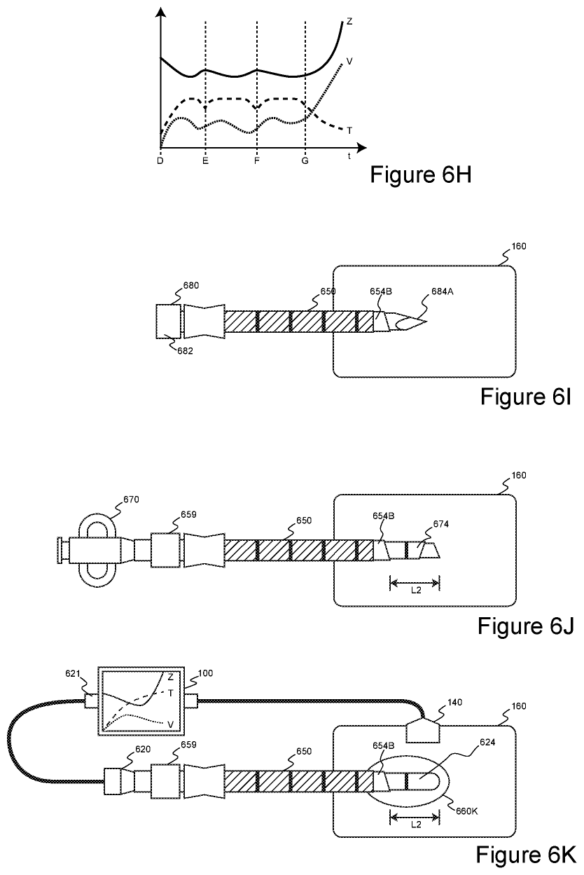

FIG. 6 refers collectively to FIGS. 6A, 6B, 6C, 6D, 6E, 6F, 6G, and 6H.

FIG. 6A presents an alternative embodiment of the present invention, which is a system comprising a cannula 650, obturating stylet 680, electrode 620, biopsy needle 670, and spacer 659. Each item is show from its proximal end (“Proximal Views” on the left”, its side (“Side Views” in the center), and its distal end (“Distal Views” on the right). The cannula 650 includes a hub 652 at its proximal end, a partially insulated shaft comprising a proximal electrically-insulated portion 654A (shown in the figure as a region filled with a diagonal line pattern) and a distal electrically-conductive portion 654B, and a lumen through the hub and shaft longitudinally. The conductive tip 654B can have a length in the range 0-5 cm or longer, including substantially the entire length of the cannula; however, inclusion of an insulated proximal shaft portion 654A has the advantage of preventing skin burn or other thermal damage during electrification of and ablation by the cannula active tip 654B, as shown for example in FIG. 6C, and furthermore, a length in the range 0.5-2 cm (e.g. 1 cm) can have special advantage as it limits generator output required for such ablation and it further limits the potential thermal damage to more superficial structures during such electrification and ablation while providing more ablation of tumor cells that might adhere to the distal cannula 654B, as described later and herein. The cannula shaft can be constructed from stainless steel hypotube, e.g. 17UTW (17 gauge, ultra thin wall), over which electrical insulation 654A is applied. The shaft 654A, 654B has depth markers 654M along its length, which can indicate intervals of length from the distal point, e.g. with 0.5 cm or 1 cm spacing. Depth marks, such as 654M and 624M, can be etched on electrically conductive tubing without substantially affecting the electrical conductivity of the marked portions of the tubing; such depth marks can be visible through transparent electrical insulation that is applied over such electrically conductive tubing. Depth marks, such as 654M, can be applied to electrical insulation itself, such as insulation 654A. Electrical insulated 654A can be low profile (e.g. 0.001-0.002″ thick with a smooth transition to the conductive tip 654B. The stylet 680 includes hub 682 at its proximal end and a solid cylindrical shaft 684A (e.g. 18G stainless steel rod) with distal tissue-piercing point 684A (e.g. trocar). The stylet shaft 684 can be inserted into lumen of cannula 650 via the proximal hole 652A of hub 652, stylet sharp distal pint 684A can extend out of cannula lumen from cannula distal opening 654C, and the hubs 652 and 682 can engage to create a solid tissue-piercing probe; and then the stylet 680 can be withdrawn to allow access to tissue by other devices, such as biopsy needle 670 and electrode 620, each of whose shafts can be similarly inserted into the lumen of cannula 650 via proximal opening 652A of its hub 652. Electrode 620 is a monopolar electrosurgical electrode (e.g. radiofrequency (RF) electrode) including a generator connector 621, hub 622 at its proximal end, and cylindrical electrically-conductive shaft 624 at its distal end. The distal point 624A of the shaft 624 is rounded. In other embodiments the distal point 624A can be sharp. Shaft 624 is conductively connected to connector 621 to connection to an electrosurgical generator (e.g. RF generator), includes depth markers 624M along its length, and can be constructed from stainless steel rod or tubing (e.g. 18 gauge (18G) hypotube). Biopsy needle 670 includes a proximal hub 672 and distal shaft 674 that includes depth markers 674M. Biopsy needle 670 can be a core biopsy needle known to one skilled in the art, such as the Bard Mission or Carefusion Temno devices. The outer shaft 674 of the biopsy device can be 18 gauge. Spacer 659 has proximal opening 659A, distal opening 659B, and can be attached to the cannula hub 652 to reduce the length by which a probe (e.g. electrode 620, biopsy needle 670) extends out from the distal opening 654C of the cannula lumen when the probe is inserted into the cannula (i.e. into the cannula lumen via its proximal hub 652) such the probe hub (e.g. electrode hub 622, biopsy hub 672) engages with the cannula hub 652. In some embodiments, the spacer 659 can has length 1 cm so that said reduction is 1 cm. In some embodiments, the multiple spacers can be include in the system to allow a physician user of the system can select the degree of said reduction by combination of spacers, or by user of spacers having different lengths.

FIG. 6B shows the biopsy needle 670 fully inserted into the cannula 650 such that their proximal hubs 672 and 652, respectively, engage. Biopsy needle 670 is inserted into cannula hub proximal opening 652A to its cannula lumen, and biopsy needle's shaft's distal end 674A extends out of the cannula lumen from cannula distal opening 654C. The length of extension L is the length by which the biopsy shaft 674 extends out from the distal opening 654C of the cannula 650. Samples from tissue 160 can be collected by the biopsy device 670 in this extension length. The extension length can be, for example, 1 cm, 2 cm, 2.5 cm, 3 cm, 4 cm, 5 cm, or >5 cm, or a value in the range 0-5 cm.

FIG. 6C shows the electrode 620 fully inserted into the cannula 650 such 650 such that their proximal hubs 622 and 652, respectively, engage. The electrode shaft 624 is in electrically-conductive contact with the inner surface of the cannula, so that when an RF generator 100 conducts a signal to the electrode shaft 624, the signal is also conducted to the cannula conductive tip 654B, thereby forming a combined active tip 654B, 624. Electrode 620 is inserted into cannula hub proximal opening 652A to its cannula lumen, and electrode's distal end 624A extends out of the cannula lumen from cannula distal opening 654C. The length of extension by which the electrode shaft 624 extends out from the distal opening 654C of the cannula 650 is the same the as the length of extension L extension by which the biopsy shaft 674 extends out from the distal opening 654C of the cannula 650. The feature that the electrode and biopsy lengths of the extension are substantially identical is an advantage because when the electrode is inserted into tissue 160 after the biopsy needle 670, then any bleeding or tumor cells along the tissue tract of the prior biopsy needle insertion (e.g. as shown in FIG. 6B) can be coagulated by delivery of electrosurgical energy, e.g. RF energy, from an RF generator 100 via the combined active tip 654B, 624 thereby creating an ablation zone 660C around the combined active tip 654B, 624. In one embodiment the generator 100 is configured to rapidly delivery energy sufficient to boil tissue around the entirely of the combined active tip 654B, 624. This has the advantage that the entire tissue tract from a prior biopsy using needle 674 can be coagulated rapid, in 15-30 seconds, without moving the insertion cannula 650. In another embodiment, the generator 100 can be configures to control a temperature or temperatures measured by the electrodes to create an temperature-controlled ablation. It is an advantage that tissue around the distal end of the cannula 654B is ablated because (1) this distal end could have been positioned among tumor cells, (2) tumor cells could be dragged into the distal cannula opening 654C by prior withdrawal of the biopsy needle 670 to collect samples from a suspected tumor, and either of the possibilities (1) and (2) could lead to spread of tumor cells when the cannula 650 is finally withdrawn from patient 160. It is an advantage that the electrode distal point 624A is rounded and the shaft 624 is stiff so that the electrode is more likely to follow the tract through tissue 160 that was formed by prior insertion of the biopsy needle 670. Another advantage of a round distal end 624A for the electrode 620 is that the distal end can be a temperature sensor located exactly at the distal end of the electrode where the tissue temperature is often highest during ablation.

The outer diameter of the conductive electrode shaft 624 and the inner diameter of the conductive cannula shaft 654B are configured to ensure conductive physical contact between the electrode shaft 624 and cannula shaft 654B, so that the ablation forms substantially outside the cannula tip 654B (i.e. not limited to the space between the electrode shaft 624 and the inner surface of the cannula lumen); this is an important feature for any of the electrode and cannula ablation systems of the present invention. For example, the difference between the outer diameter of the electrode shaft 624 and the inner diameter of the cannula shaft 654B can be less than 0.010″. In some embodiments, the electrode shaft 620 can be bent to ensure physical contact with the cannula shaft 654B. In some embodiments of electrode 620 and cannula 650, as well as of other electrode-cannula systems set forth herein in which conductive surfaces of the electrode and cannula are conductively connected to be at the same potential, other structures and methods can be used for such conductive connection, including complementary contacts within the electrode and cannula hubs.

FIGS. 6D, 6E, 6F, 6G show a sequence in which the electrode 620 is withdrawn slowly, e.g. over a period of 15-30 seconds, while generator 100 delivers ablation energy between electrode 620 and ground pad 140, to rapidly create an ablation zone. In one embodiment of this example, the generator 100 can be configured to control the temperature measured at the distal point 624A of the electrode 620. In FIG. 6D, the electrode 620 is fully inserted into the cannula 650, energy is applied, that the ablation 660D forms predominantly at the distal end 624A of the electrode, which is typical for ablation electrodes, particularly when the tip length is longer, e.g. greater than or equal to 2 cm. The ablation zone around the cannula active tip 654B may be more narrow at this time. Once the electrode temperature reaches a target value (e.g. 70-90+degC), the electrode 620 is withdrawn partially within the lumen of the cannula 650, such that the cannula location is unchanged to achieve the position shown in FIG. 6E. As the electrode temperature sensor may have been drawn into cooler tissue, the electrode temperature (T) measured by the generator 100 may drop initially, and then recover as the generator 100 increased the output voltage (V) to re-achieve a temperature set value and the ablation zone 660E thickens around the new location of the electrode distal end. This withdrawal process may be continuous or repeated one or more times to achieve the configuration in FIG. 6F wherein the electrode and cannula distal ends are aligned, the cannula electrically-conductive portion 654B is now the predominance of the combined active tip 654B, 624, and the ablation zone 660F thickens around the cannula active tip 654B, thus creating an ablation zone of relatively uniform thickness around the extension length where the biopsy needle 680 previously cut through tissue, which tissue may have included cancerous cells. The coagulation can stop at this point, for example, by means of observing a depth marker or markers 624M along the proximal electrode shaft that has been withdrawn proximally from the cannula hub 652; or, alternatively, the electrode 620 can be further withdrawn by the user so that its distal end 624A is within the insulated portion of the cannula shaft 654A as shown in FIG. 6G (in this figure the portion of the electrode shaft 624, including its distal end 624A, that is within the lumen of cannula 650 is shown as a dotted line), so that the measured electrode temperature drops (because tissue around the electrically-insulated shaft 654A is not heated), the generator increases generator output (V) in a futile attempt to achieve a target temperature, and tissue around the cannula active tip 654B boils, causing a large increase in the electrical impedance (Z) measured by the generator 100, an enlarged ablation zone 660G around the cannula active tip 654B, and signaling to the user or to the generator that the ablation process is complete. It is an advantage to no move the cannula 650 during biopsy and ablation because it simplifies the post-biopsy ablation process.

FIG. 6H shows generator readings for electrode temperature (T), generator output (V) (for example Voltage, Current, or Power), and tissue impedance (Z) graphed over time axis (t) during the process of FIGS. 6D through 6G, wherein the time axis is marked with times D, E, F, G corresponding to the configuration of FIGS. 6D, 6E, 6F, and 6G, respectively.

FIG. 6I shows a combination of the cannula 650 and stylet 680 that can be used to insert the cannula 650 into tissue 160, wherein the stylet 680 has been inserted into the cannula lumen via its proximal opening 652A such that the hubs 682 and 652 engage and the sharp distal point 684A of the stylet 680 protrudes from the distal end of the cannula 654A through opening 654C. The stylet 680 can then be removed and the biopsy needle 670 can be inserted into the tissue 160 via the cannula 650; in some embodiments of this operation, as shown in FIG. 6J, the spacer 659 is first attached to the hub 652 of the cannula 650, and then the biopsy needle 670 is inserted into the proximal hole 659A of the spacer, traverses the spacers lumen, exits the spacer's distal hole 659C into cannula lumen via cannula's proximal hole 652A, traverses the cannula lumen, and exits the cannula's distal hole 654C into tissue 160 with extension length L2 beyond the cannula distal opening 654C. The biopsy needle 670 can then be withdrawn from the combination of the cannula 650 and spacer 659, and it can be replaced by electrode 620 as shown in FIG. 6K, wherein the extension length of the electrode 620 beyond the cannula is the same length L2. In other embodiments, the cannula insertion of FIG. 6I can precede the biopsy needle insertion of FIG. 6B, which can subsequently precede the electrode insertion of FIG. 6C. In other embodiments, multiple spacers 659 can be stacked onto the cannula 650 and onto each other to create different extensions lengths. In other embodiments, the extension lengths of the electrode and the biopsy needle can be different, for example, to suit clinical needs. In some embodiments, the extension length of the electrode can be longer than that of the biopsy needle (e.g. the sequence FIG. 6I, FIG. 6J, FIG. 6C), for example, to ensure that tissue distal to the biopsy is coagulated, or to ablate a target structure (such as a tumor) to which a biopsy sample was collected centrally. In some embodiments such use of a spacer as shown in FIG. 6J is used for a “short throw” more superficial tissue sample collection (e.g. 10-15 mm extension length), as opposed to a “long throw” deeper biopsy sample collect (e.g. 20-25 mm extension length).

In some embodiments, the cannula 650, stylet 680, spacer 659, and/or electrode 620 can be sized for compatibility with one or more separately provided biopsy devices 670, such as those sold by a variety of manufacturers.

The system of electrode 620 and cannula 650 has special advantage for track coagulation because the entirety of the extension length of the electrode is electrically activated and heating tissue throughout withdrawal of the electrode 620 into the cannula 650. Thus, even if the rate of heating is greater at the distal end of the monopolar combined active tip 654A, 624, the more proximal aspects of the combine tip are heated throughout. This helps to ensure that gaps in the ablation zone are not created by too fast withdrawal of the electrode into the cannula.

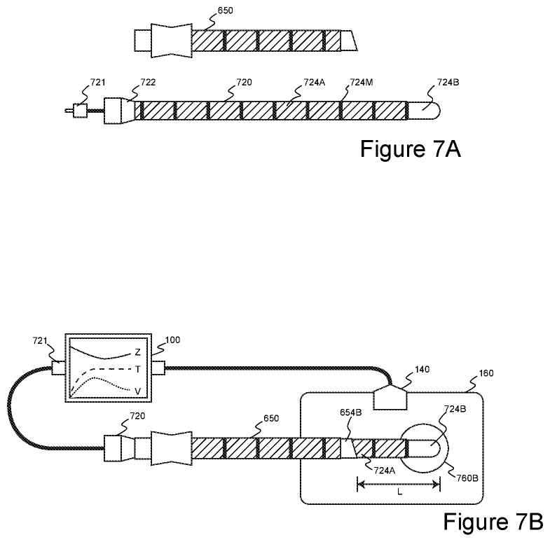

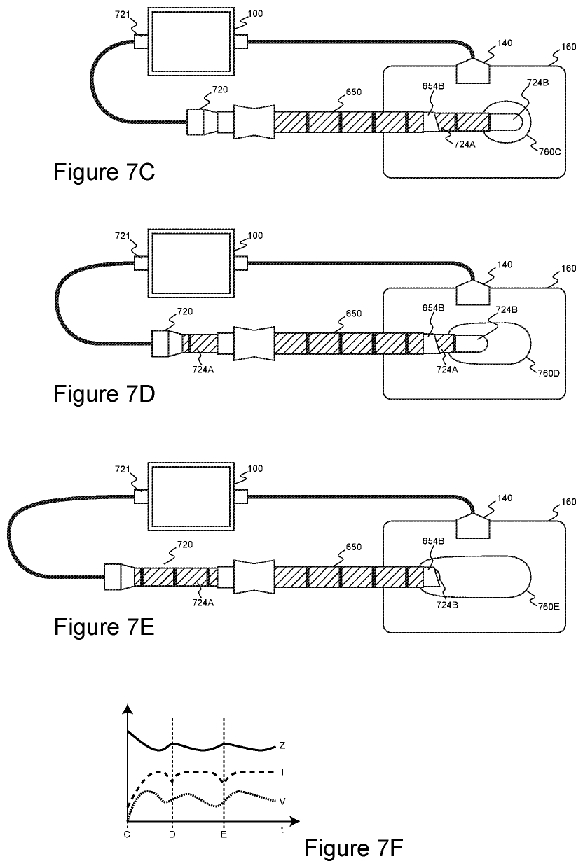

FIG. 7 refers collectively to FIGS. 7A, 7B, 7C, 7D, 7E, and 7F.

In FIG. 7A, electrode 620 of FIG. 6A is replaced by monopolar electrode 720, which has the additional feature that its proximal shaft 724A is electrically insulated (shown in the figure as a region filled with a diagonal line pattern), and has an electrically conductive distal end 724B. In one example, electrode 724A can be constructed from 19-gauge stainless steel rod or hypotube over which electrical insulation 724A is applied so that it fits through a 17UTW shaft of cannula 650. The shaft of electrode 720 has depth marks 724M along its length.

FIG. 7B shows electrode 720 inserted into cannula 650 with the same extension length L and producing an ablation zone 760B around the active tip 724B. The electrical insulation 724A prevents conduction of the generator output to cannula tip 654B.

FIGS. 7C, 7D, 7E, show the process of withdrawing the electrode 720 into the cannula 650 without moving the cannula while the generator 100 delivers electrical energy to the electrode 620 using ground pad 140 to carry return current. The ablation zone 660C extends as the electrode tip 724B is dragged proximally toward the cannula, until ultimately the electrode conductive tip 724B contacts and electrifies the cannula conductive tip 654B in FIG. 7E thereby extending the ablation zone 660E around the cannula active tip 654B.

FIG. 7F shows generator readings for electrode temperature (T), generator output (V), and tissue impedance (Z) graphed over time axis (t) during the process of FIGS. 7C through 6E, wherein the time axis is marked with times C, D, E corresponding to the configuration of FIGS. 6C, 6D, 6E, respectively.

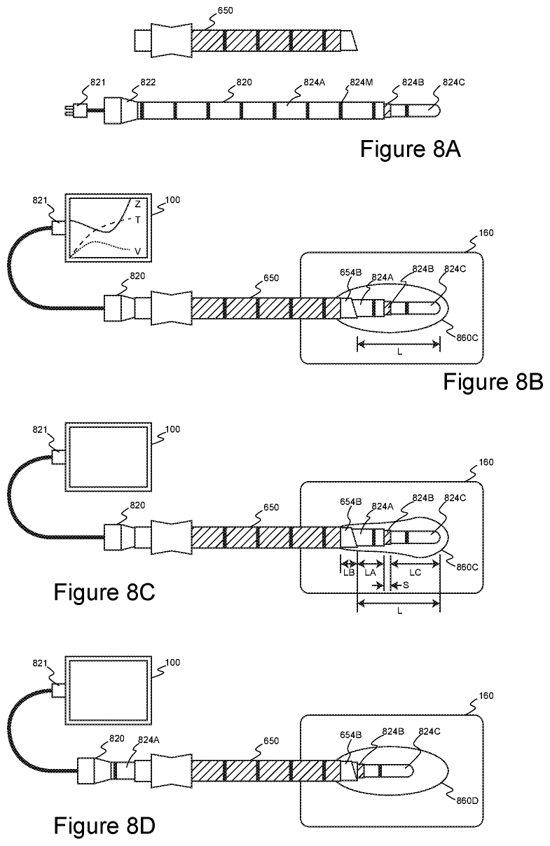

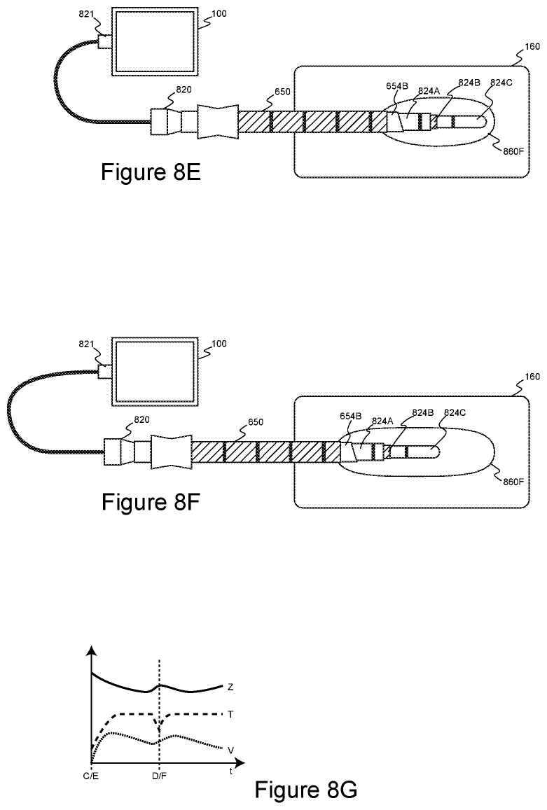

FIG. 8 refers collectively to FIGS. 8A, 8B, 8C, 8D, 8E, 8F and 8G.

In FIG. 8A, electrode 620 of FIG. 6A is replaced by bipolar electrode 820, which has the additional features that is connector 821 conduct both output poles (e.g. high voltage and reference) of generator 100 to the electrode shaft. In particular, one pole is conducted to the proximal conductive shaft 824A, the other pole is conducted to the distal conductive shaft 824C, and the two conductive shaft portions 824A and 824C are electrically insulated with in the electrode and are separated by intermediate electrically-insulative shaft portion 824B. In some embodiments, the distal tip 824C can be constructed from stainless steel rod or hypotube, the insulated inter-tip regions 824B (shown in the figure as a region filled with a diagonal line pattern) can be electrical insulation applied over said rod or hypotube, and the proximal tip 824A can be stainless steel hypotube applied over the electrical insulation, e.g. 824A can be 18UTW hypotube and 824C can be 20 gauge rod or tubing and can fit within an 17UTW shaft for cannula 650.

FIG. 8B shows electrode 820 inserted into cannula 650, positioned in tissue 160. Electrode proximal tip contacts the conductive inner lumen of the cannula 650 so that the signal from electrode tip 824A also conducts through cannula tip 654B to form a combined proximal tip 654B, 824A. Electrode 820 is electrified by generator 100 in a bipolar configuration whereby current flows from combined tip 824C, 854B to electrode tip 824C through tissue 160 to form ablation zone 860B. The inter-tip region is configured to be short enough longitudinally (e.g. 2 mm, or a length in the range 1-5 mm) so that the ablation zone is confluent between the active tips 824C and 824A, 854B. In one embodiment the generator 100 is configured to rapidly delivery energy sufficient to boil tissue around the entirely of the shaft portion spanned by 654B, 824A, 824B, 824C. This has the advantage that the entire tissue tract from a prior biopsy using needle 674 can be coagulated rapid, in 15-30 seconds, without moving the insertion cannula 650. In another embodiment, the generator 100 can be configured to control a temperature or temperatures measured by the electrode to create an temperature-controlled ablation. The extension length L for electrode 820 is the same as for the biopsy needle 670 in FIG. 6A.

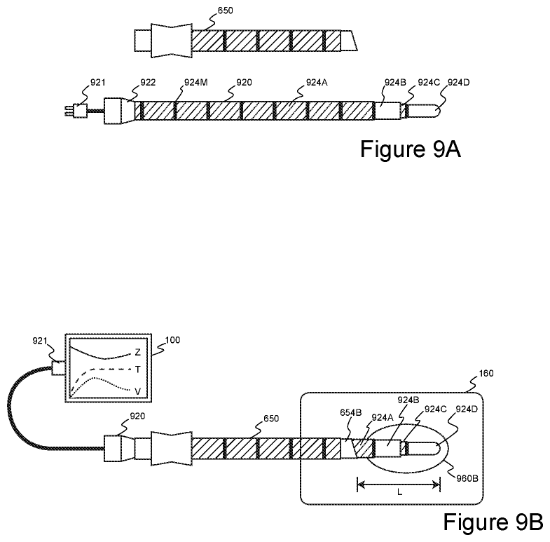

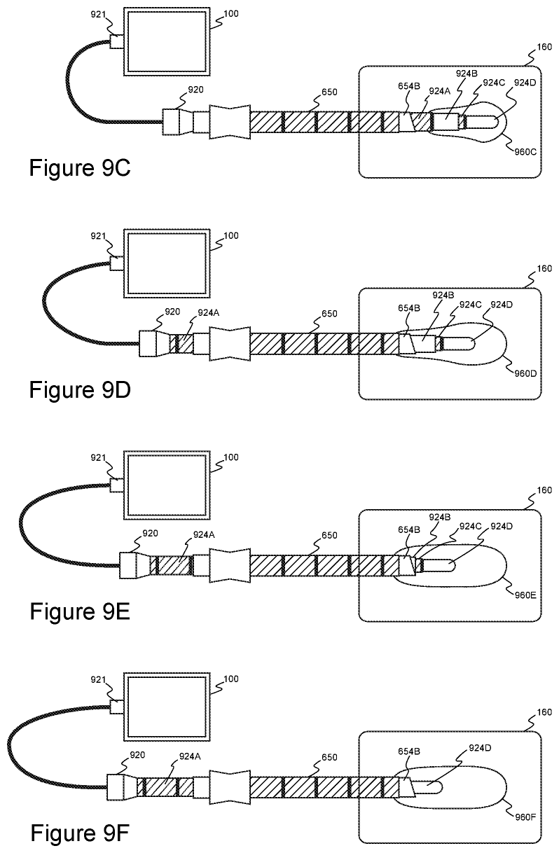

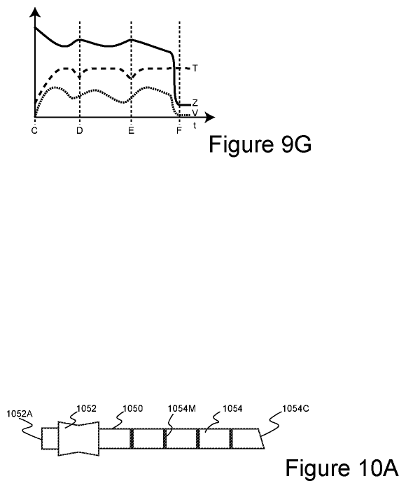

The cannula 650 and electrode 820 can be configured so that the surface area of the following elements is strictly increasing: cannula tip 654B; distal electrode tip 824C; combined active tip 654B, 824A when the electrode 820 is fully inserted into the cannula 850 so that their hubs engage. This has the advantage that when the electrode is fully inserted into the cannula 650 (as in FIG. 8C), the current density, tissue heating, and thus the ablation zone 880C is focused toward the smaller distal active tip 824C. This distal focus continues to be the case as the electrode 820 is withdrawn in to the cannula 850 while delivering ablation energy from generator 100, until the proximal electrode tip 824A becomes sufficiently shrouded in the cannula lumen that the ablation the cannula active tip 854B is substantially all of the proximal active tip, and thus the current density, tissue heating, and thus the ablation zone 880D becomes focused toward the smaller proximal active tip 854B (and, in some cases, some of the remaining non-shrouded electrode proximal shaft 824A, thereby creating a uniform ablation zone around the extension length. This ordering of relative tip length can be applied to other bipolar electrode/cannula systems set forth herein, including that of FIGS. 9A-F, 17C, 17D. In some embodiments, other features of the cannula 650 and electrode 820 can be configured to ensure that (1) when the distal electrode tip 824C and combined proximal electrode/cannula tip 654B, 824A are energized with different potentials, heating of tissue at and around the distal electrode tip 824C is more rapid than around the combined proximal electrode/cannula tip 654B, 824A, and/or (2) when the distal electrode tip 824C and the cannula active tip 854B (alone) are energized with different potentials, heating of tissue at and around the cannula active tip 854B is more rapid than around the distal electrode tip 824C, in order to ensure the formation of uniform ablation zone 880D when energy is applied to the electrode 820 as it is withdrawn within the cannula 650: For example, the relative length of the tips (e.g. for uniform outer diameter of these tips, the relative length), the relative construction of the tips, the relative heat conduction/sink rate of each of the tip (e.g. due to different metallic shaft wall thicknesses, the presence of coolant).

In one example, the cannula active tip 654B is 17UTW, has an outer diameter of 0.058″, a length of LB=5 mm, and a surface area of 0.29π in-mm; the proximal electrode tip 824A is 18UTW, has outer diameter 0.050″, has maximal protrusion length LA=8 mm, and a surface area of 0.40π in-mm; the combined surface area of the cannula tip 654B and maximally extended proximal electrode tip 824A is (0.29+0.40)π=0.69π in-mm; the inter-tip spacing 824B has length S=2 mm; the distal electrode tip 824C is 20G, has outer diameter 0.036″, has length LC=15 mm, and has surface area 0.54π in-mm.

In some embodiments of the bipolar electrode systems of FIGS. 8B, 9B, 16C, 16D, 17C, 17D, 18B, 19B, configuring the more distal (combined) active tip to be smaller (or more rapidly heating) than the more proximal active tip can be used to ensure heating around the distal end of bipolar probe. In some embodiments of the bipolar electrode systems of FIGS. 8B, 9B, 16C, 16D, 17C, 17D, 18B, 19B, configuring the more distal (combined) active tip to be larger (or less rapidly heating) than the more proximal active tip can be used to ensure heating around the proximal active tip. In some embodiments of the bipolar electrode systems of FIGS. 8B, 9B, 16C, 16D, 17C, 17D, 18B, 19B, configuring the inter-tip region to be small (e.g. 2 mm, or a length in the range 1-5 mm) can be an advantage to create a confluent ablation zone between and around both bipolar tips, because a small gap will focus the current density and tissue heating between the two bipolar tips; once that region reaches boiling, the impedance increases there and current flows to more separated portions of the bipolar tips, thereby creating an increasing elongated ablation zone in the direction of the bipolar shaft, until the ablation zone reaches the most distant aspect of the one (or both) of the bipolar tips that is closest to the center (or that heats more preferentially). Such propagating boiling can be advantage to reliably generating an ablation zone around two bipolar active tips on a single electrode shaft.

FIGS. 8E and 8F show an alternative example wherein the electrode 820 and cannula 650 are withdraw as a unit to form an elongated ablation zone. This can apply to any of the electrode 620, 720, 820, and 920 in FIGS. 6, 7, 8, 9.

FIG. 8G shows generator readings for electrode temperature (T), generator output (V), and tissue impedance (Z) graphed over time axis (t) during the process of FIGS. 8C and 8D, or of FIGS. 8E and 8, wherein the time axis is marked with times C/E and D/F, corresponding to the configuration of FIG. 8C or 8E, and 8D or 8F, respectively.

The system including the electrode 820 and cannula 650 have special advantage because the entire extended electrode length and cannula active tip are heating through the process of withdrawing the electrode 820 into the cannula during track ablation. This help avoid gaps in the ablation zone due to too fast withdrawal of the electrode during track ablation even if tissue heating is biased toward the distal end of the electrode due to relative geometry of the electrode and cannula active tip, because tissue heating at a lower level proceeds at more superficial/proximal aspects of the track for a sustain duration (i.e. throughout the withdrawal process).

FIG. 9 refers collectively to FIGS. 9A, 9B, 9C, 9D, 9E, and 9F.

In FIG. 9A, the electrode 820 of FIG. 8A is replaced by bipolar electrode 920 which has the additional feature of a proximal electrical insulated region 924A (shown in the figure as a region filled with a diagonal line pattern) over is proximal shaft. Electrode 920 has shaft portions 924A electrically insulated, 924B electrically conductive and connection to one generator pole via connector 921, 924C electrically insulated inter-tip portion (shown in the figure as a region filled with a diagonal line pattern), and 924D electrically conductive and connected to the other generator pole by connected 921.

FIG. 9B shows electrode 920 fully inserted into cannula 650 so their hubs engage, positioned in tissue 160, and electrified by generator 100 to produce ablation 960B in tissue 160 due to current flowing through the tissue 160 from electrode tip 924D to 924B. Cannula tip 654B is not electrified in this configuration due to electrical insulation 924A. The extension length L of electrode 920 matches that for biopsy needle 670. In some embodiments the distal tip can be 21 or 22 gauge stainless steel rod or tubing, the proximal tip can be 19UTW stainless steel hypotube, so that electrode 920 fits within a cannula 650 shaft constructed from 17UTW hypotube.

FIG. 9C through 9F show the process of withdrawing electrode 920 into cannula 850 while delivering bipolar ablation energy between the electrode active tips (and the cannula active tip 854B when in FIGS. 9D and 9E the proximal electrode tip 924B enters the cannula lumen). In FIG. 9F, the distal electrode tip 924D enters the cannula lumen creating a short circuit (low impedance) which can be detected by the generator 100 and/or the user as a useful endpoint to the tract ablation process; this can apply to FIG. 8 as well. The electrode distal tip 924D can be configured to have smaller surface area (or, more generally, to heat more rapidly) than the electrode proximal tip 924B, so that tissue heating is preferential to, and the ablation zone 960C extends over the entire, distal electrode tip 924D (and in particular distal to it) while the proximal electrode tip 924B is extended into tissue beyond the cannula distal end 654B (as in FIG. 9C), and also while the combination of the proximal electrode tip 924B and cannula tip 654B are in conductive contact to create an combined tip 924B, 654B with an even larger surface area (and thus less current density and thus less relative propensity for heating) as shown in FIG. 9D and by ablation zone 960D. It is advantage to bias the heating toward the distal tip to ensure complete ablation of the entire tissue tract formed by the biopsy needle previously inserted through the cannula, including at the farthest point form the cannula distal end 654B. Advantageously, even with such distal heating bias, withdrawal of the electrode 920 into the cannula can still produce some coagulation at the distal end of the cannula 654B because the cannula is immobile, carrying some electrical current, and thus heating tissue throughout the electrode withdrawal; thus by carrying lower current in one location for a longer duration, tissue temperature around the cannula active tip 654B can reach destructive levels. However, additionally, the cannula active tip 654B can be configured to have smaller surface area than that of the electrode distal tip 924D, so that once the proximal electrode tip 924B is sufficiently shrouded within the cannula lumen, the cannula tip 654B will heat more rapidly than the electrode distal tip 924D and the ablation zone 960E will extend more strongly around the cannula active tip 654B as shown in FIG. 9E to create a confluent ablation zone of sufficient and/or uniform thickness along the length of the tissue tract of the previously inserted biopsy needle 670.

In one example, the cannula active tip 654B is 17UTW, has an outer diameter of 0.058″, a length of 5 mm, and a surface area of 0.29π in-mm; the proximal electrode tip 924B is 19UTW, has outer diameter 0.042″, length 8 mm, and a surface area of 0.34π in-mm; the maximal combined surface area of the cannula tip 654B and proximal electrode tip 924B is (0.29+0.34)π=0.63π in-mm; the inter-tip spacing 924C is 2 mm long; the distal electrode tip 924D is 21G, has outer diameter 0.032″, length 10 mm, and has surface area 0.32π in-mm.

Electrode and cannula systems (such as the one shown in FIG. 9) that include (1) a cannula having an distal active tip 654B and proximally insulated shaft 654A, and (2) an bipolar electrode 920 having a proximally insulated shaft 924A whose overall shaft length is matched to a biopsy needle 670, have substantial advantages for needle track coagulation, particular after biopsy. These advantages include, without limitation: (1) ease of not using a ground pad 140 due to bipolar operation; (2) safety of preventing skin burns and damage to more superficial structures due to proximal cannula shaft insulation 654A; (3) safety of preventing skin burns and damage to more superficial structures even if the electrode 920 is used with an uninsulated cannula (e.g. 1050) due to the electrode's proximal shaft insulation 924A; (4) ability to create rapidly confluent ablation zone both around the tissue track from prior biopsy needle insertion via the cannula 650 and around the distal end 654B of the cannula where biopsied tumor tissue could collect (e.g. 960F) by withdrawal of the electrode into the cannula 650 (particularly when the relative size of the cannula 654B and electrode tips 924D and 924B as described in the previous paragraph); (5) a detectable stopping condition for withdrawal by means of short circuit detection when the distal electrode tip 924D contacts the cannula tip 654B (which is in conductive contact with the proximal electrode tip 924B) as shown in FIG. 9F; (6) electrode and cannula tip lengths can be configured to handle both “short throw” (e.g. 10 mm) or “long throw” (20 mm) extension a core biopsy device through a cannula; (7) lower power requirement for generator due to bipolar operation rather than monopolar operation.

FIG. 9G shows generator readings for electrode temperature (T), generator output (V), and tissue impedance (Z) graphed over time axis (t) during the process of FIGS. 9C through 9F, wherein the time axis is marked with times C through F corresponding to the configuration of FIGS. 9C through 9F, respectively.

Electrode and cannula systems (such as those shown in FIGS. 6, 7, 8, and 9) that include (1) a cannula having an distal active tip 654B and proximally insulated shaft 654A, and (2) an electrode 620,720,820, or 920 whose overall shaft length is matched to a biopsy needle 670, have substantial advantages for needle track coagulation, particular after biopsy. These advantages include, without limitation: (1) safety of preventing skin burns and damage to more superficial structures due to proximal cannula shaft insulation 654A; (4) ability to create rapidly a confluent ablation zone both around the tissue track from prior biopsy needle insertion via the cannula 650 and around the distal end 654B of the cannula where biopsied tumor tissue could collect (e.g. 960F) by withdrawal of the electrode into the cannula 650; (6) electrode and cannula tip lengths can be configured to handle both “short throw” (e.g. 10 mm) or “long throw” (20 mm) extension a core biopsy device through a cannula 650.

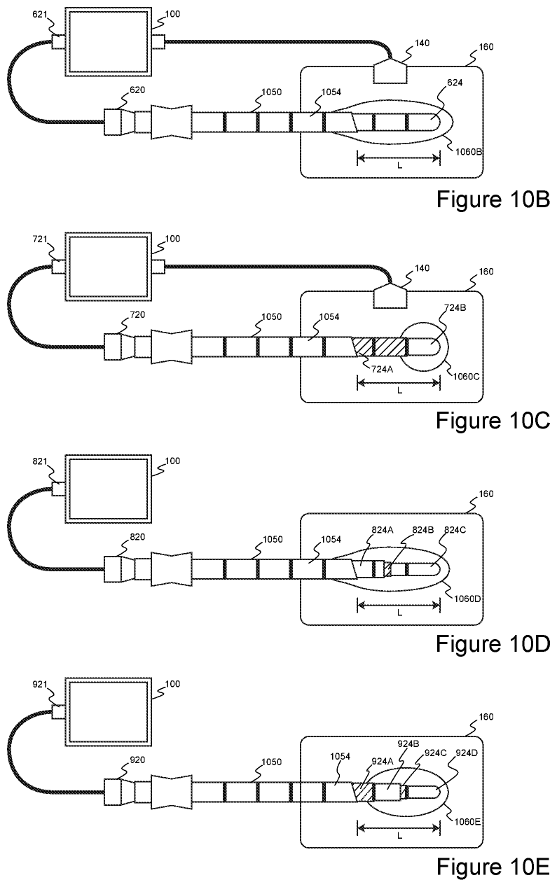

FIG. 10 refers collectively to FIGS. 10A, 10B, 10C, 10D, and 10E.

In FIG. 10A, the cannula 650 in the system of FIG. 6A is replaced by cannula 1050, whose shaft 1054 is electrically conductive and does not include electrical insulation. Cannula 1050 includes hub 1052 at its proximal end, shaft 1054 which can be composed of stainless steel hypotube, depth markers 1054M which index the distance along the shaft 1054 from its distal point, and a lumen extending from proximal hub hole 1052A through the hub 1052 and shaft 1054 to distal shaft opening 1054C. Cannula 1050 can have the same geometry as, and interaction with biopsy needle 670 as, cannula 650, including extension length L.

FIG. 10B shows electrode 620 inserted into cannula 1050 and electrified in a monopolar configuration by generator 100 to form ablation zone 1060B in tissue 160. The combination of the cannula 1050 and electrode 620 form a singular electrosurgical probe by conductive contact between the inner lumen of the cannula 1050 and the outer surface of electrode shaft 624.

FIG. 10C shows electrode 720 inserted into cannula 1050 and electrified in a monopolar configuration by generator 100 to form ablation zone 1060C in tissue 160. Electrode shaft insulation 724A prevents electrification of and ablation around the cannula shaft 1054, unless electrode active tip 724B is withdrawn into the cannula shaft 1054, in which case the combination of the cannula 1050 and electrode 720 form a unitized electrosurgical probe by conductive contact between the inner lumen of the cannula 1050 and the outer surface of electrode shaft 724B.

FIG. 10D shows electrode 820 inserted into cannula 1050 and electrified in a bipolar configuration by generator 100 to form ablation zone 1060D in tissue 160. The combination of the cannula 1050 and electrode 820 form a singular bipolar electrosurgical probe by conductive contact between the inner lumen of the cannula 1050 and the outer surface of the proximal electrically conductive electrode shaft portion 824A.

FIG. 10E shows electrode 920 inserted into cannula 1050 and electrified in a bipolar configuration by generator 100 to form ablation zone 1060E in tissue 160. Electrode shaft insulation 924A prevents electrification of and ablation around the cannula shaft 1054, unless electrode active tip 924B is withdrawn into the cannula shaft 1054, in which case the combination of the cannula 1050 and electrode 920 form a unitized electrosurgical probe by conductive contact between the inner lumen of the cannula 1050 and the outer surface of electrode shaft 924B.

Referring to FIGS. 10B-10E, the extension length L for each electrode 620, 720, 820, 920 is substantially similar to that for biopsy needle 670 when inserted through cannula 1050. Though the cannula shaft 1054 can be wholly electrified by contact between cannula lumen and the electrode's shaft (which occurs in the configurations of FIGS. 10C and 10E if electrode active tips 724B and 924B, respectively, are withdrawn into cannula shaft 1054), the ablation zones (1060B, 1060C, 1060D, 1060E) forms primarily at the distal end of the combined shaft of the cannula and electrode due to the geometry of the electric field being highest at the electrode distal point. However, there is risk of skin burn at the surface of tissue 160 due to elevated electric fields at that location as well as along the entirety of the cannula shaft 1054, particularly if the depth of insertion of the cannula 1050 into the tissue 160 is shallow. An advantage of an uninsulated cannula 1050 is that is more simple, less expensive, and more readily available than an insulated cannula (such as 650); however, a partially insulated cannula having an electrically non-conductive proximal shaft portion (e.g. 650) has the advantage of reducing the risk of thermal damage to non-target structures (such as the skin, organs, and biological divisions such as the chest wall) along the proximal length of the cannula shaft and allows the physician both to control thermal damage to more superficial non-target structures during ablation and to ablate tissue around the distal opening of the cannula (e.g. 654C).

One embodiment of the present invention is a system that includes an ablation electrode (e.g. 1120, 1220, 1320, 1420) that is compatible with a biopsy device (e.g. an electrode that fits through the lumen of the coaxial introducer needle 1150 for a core biopsy device 1170) and an electrosurgical generator 1100 (e.g. a radiofrequency generator) having temperature and/or impedance displays (e.g. numeric, time-dependent line graphs, dynamic bar graphs, dynamic graphs showing the current and a minimum and/or maximum value, dynamic meters, and other graphical displays) for an ablation electrode. In one method of the present invention:
A biopsy device 1170 is introduced through an introducer 1150 and a target tissue 1169 (e.g. a suspected tumor) in a living body 1160 (e.g. a human or animal patient) is biopsied using the biopsy device
The biopsy device is withdrawn from the introducer 1150
An ablation electrode (e.g. 1120, 1220, 1320, 1420) is introduced through the introducer 1150 substantially along the same tract through the tissue 1160 as the biopsy device 1170. In some embodiments of this step, the distal end of the ablation electrode stops at substantially the same location as the point of deepest insertion of the biopsy device 1170. In some embodiments of this step, the distal end of the ablation electrode stops beyond the point of deepest insertion of the biopsy device 1170.
The generator 1100 delivers high-frequency output (e.g. monopolar RF, bipolar RF, MW) to the bodily tissue 1160 via the ablation electrode, and the generator 1100 displays at least one measurement related to tissue heating near the ablation electrode (e.g. temperature or impedance), while the user withdraws the ablation electrode from the tissue 1160, thereby coagulating the tissue around the said tract. The purpose of said coagulation can be the thermal destruction (by means of coagulation, also known as ablation) of tumor cells that may have been carried along with the biopsy device 1170 during its withdrawal). The purpose of said coagulation can be the stoppage of bleeding from blood vessels damaged during the insertion of the biopsy device 1170.

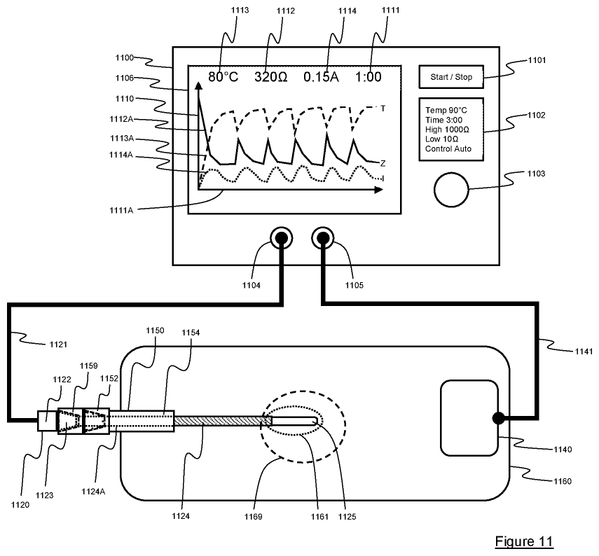

FIG. 11 shows one embodiment of the present invention that includes an RF generator connected to monopolar electrode 1120 which is inserted into body 1160 via introducer needle 1150 and spacer 1159 along the same tissue tract previously traversed and/or produced by the insertion of a biopsy device through cannula 1150. Cannula 1150 includes a cylindrical shaft 1150 with lumen therethrough and a hub 1152 having a proximal female luer opening. Spacer 1159 includes male luer that interlocks with the female luer of the introducer hub 1152. Electrode 1120 includes cable 1121 to the generator 1100, hub 1122, coupling feature 1123 (e.g. a male luer that couples with the spacer 1159 or the cannula hub 1152), shaft insulated portion 1124 (which is shown as a dotted line 1124A within the lumen of the cannula shaft 1154), shaft uninsulated portion 1125 at the electrode distal end. The insulated shaft 1124 is cylindrical and electrically non-conductive, and it prevents or substantially limits the flow of RF signal output from the generator 1100 to tissue 1160. The exposed active tip 1125 is cylindrical, metallic, and conductive, and it conducts RF current from the generator 1100 via cable 1121 to the tissue 1160. The distal point of the electrode tip 1125 is rounded. One advantage of a rounded distal tip is that it tends to follow a tissue tracts previously cut through the tissue 1160 by a biopsy device so that ablation produced by the electrode 1120 is more certain to include the tissue cut and traversed by that previous insertion of the biopsy device. In another embodiment, the electrode can have a sharp distal point. Ground pad 1140 is applied to the skin surface of body 1160 and carries return current from active tip 1125 to the generator 1100 via cable 1141. The RF output through tip 1125 produces tissue heating around the tip 1125, which heating is shown as the coagulation zone 1161, which is axially symmetric around the electrode shaft (which comprises insulated portion 1124 and active tip portion 1125). The electrode cable 1121 connects to a first output jack 1104 of the generator. The ground pad cable 1141 connects to a second output jack 1105 of the generator. The generator produces an RF potential across pins of the jacks 1104 and 1105. The generator 1100 includes a Start/Stop toggle button 1101 for starting and stopping the RF output to connected electrodes and ground pads. The generator 1100 includes user-adjustable settings 1102. In some embodiments, the settings 1102 can include some or all of a target temperature, a maximum output-on time, high and low limits on the electrode impedance, and selection of manual or automatic control. In some embodiments, the generator settings can include target voltage, current, and/or power settings. The generator includes a knob 1103 for the option of manual control of the generator output level. The generator display 1106 includes numeric display of electrode and generator measurements, including the electrode temperature 1113 (that is measured using a temperature sensor include in the electrode tip 1125), the impedance between the electrode 1120 and the ground pad 1140, the generator output level 1114 (which in this embodiments is RF current, and can be voltage, power, and/or current in other embodiments), and the elapsed output-on time 1111. In this embodiment and mode of the generator display 1106, the temperature 1113, impedance 1112, and current 1114 are each graphed on vertical axis 1110 as a function of the axis 1111A for the time 1111; the respective graphs being 1113A for temperature 1113, 1112A for impedance 1112, and 1114A for current 1114. The user can monitor these measurement displays as the electrode 1120 is withdrawn from the tissue in order to produce a confluent ablation zone 1161 along the tract of the biopsy device, also known as a tract burn. Such monitoring of temperature and/or impedance helps reduce the likelihood of producing gaps in the ablation zone that could leave living tumor cells as a result of the user's withdrawing the electrode 1120 too quickly and thereby not adequately heating and coagulating the entire length of the tract.

FIG. 12 shows another embodiment of the present invention in which the monopolar electrode 1120 has been replaced by a bipolar electrode 1220. The bipolar electrode includes generator cable 1221, hub 1220, male luer 1223, proximal insulated shaft 1224A (that appears as dotted line 1224AA in the lumen of introducer 1150), proximal electrode contact 1225A, distal insulated shaft portion 1224B, and distal electrode contact 1225B. The electrode shaft comprises 1224A, 1225A, 1224B, and 1225B and is substantially cylindrical. Electrode 1220 is a bipolar electrode wherein generator RF output flows between electrodes 1225A and 22B through the tissue 1160. A ground pad 1140 is omitted relative to FIG. 11. The said flow of RF current generated tissue heating around and between tips 1225A and 1225B thereby producing ablation zone 1261, which can be substantially axially symmetric provided RF energy has been delivered a for sufficiently long time, e.g. 15-30 seconds or more. In the embodiment shown in FIG. 12, the display 1106 of generator 1100 replaces the line graphs of FIG. 11 with dynamic graphs. The first graph 1213 plots the temperature reading 1113 as line 1213A and plots the maximum measured temperature as line 1213B. Line 1213A can be a bar graph in other embodiments. The second graph 1212 plots the impedance reading 1112 as line 1212A and plots the minimum measured impedance as line 1212B. Line 1212A can be a bar graph in other embodiments. In other embodiments, other readings can be plot using dynamic bar graphs and other similar graphs such as those shown in FIG. 12, and other maximum, minimum, and/or average values can be plotted in additional to the real-time readings. In some embodiments, minimums and maximums of impedances, temperatures, and output levels can be displayed numerically and in other graphical forms.

In other embodiments, the line graphs of FIG. 11 can be used for a bipolar electrode, such as electrode 1220. The graphs of FIG. 12 can be used for a monopolar electrode, such as electrode 1120.

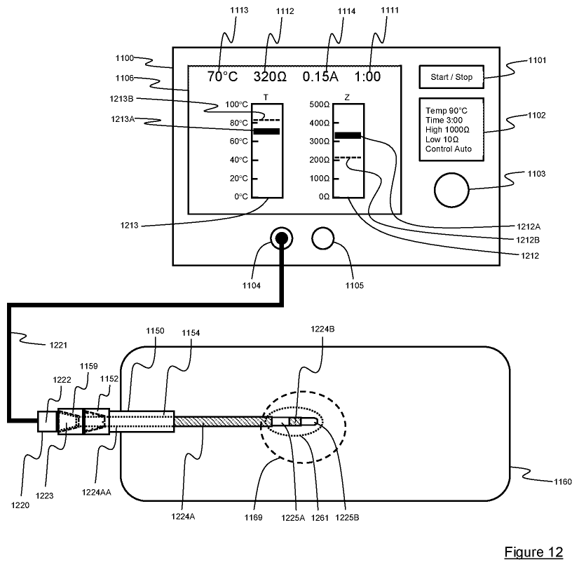

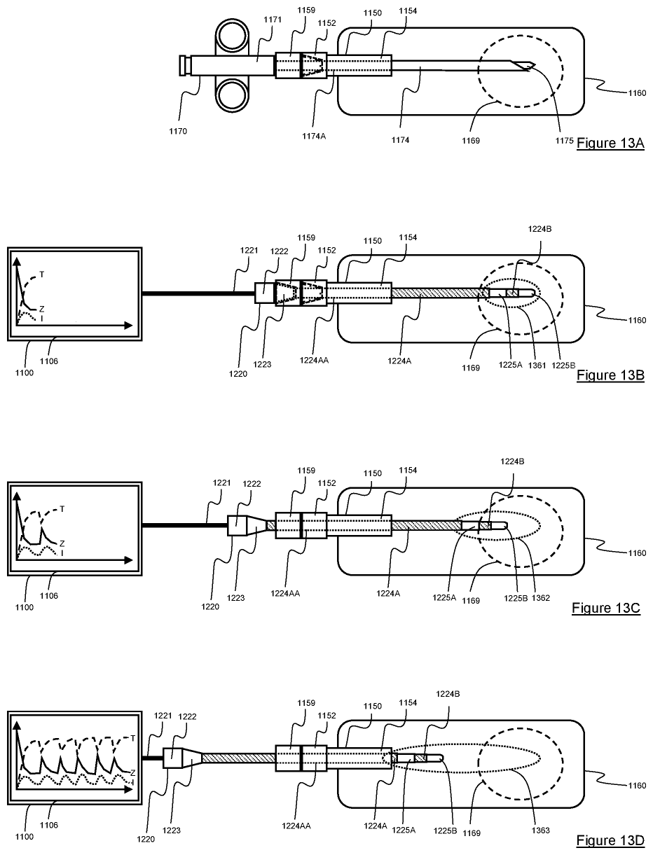

FIG. 13 refers collectively to FIGS. 13A, 13B, 13C, and 13D. FIG. 13 shows the process of producing a tract burn after tissue biopsy in accordance with the present invention. In FIG. 13A, a biopsy device 1170, such as a coaxial biopsy device is introduced into tissue 1160 through introducer 1150 and spacer 1159, a biopsy sample is collected from tumor 1169, and then the biopsy device 1170 is withdrawn from introducer 1150. Biopsy device 1170 includes a handle 1171, outer shaft 1174 comprising hypodermic tubing, and an inner biopsy stylet with sharp distal point 1175.

In FIG. 13B, the electrode 1220 is inserted through introducer 1150. Electrode 1220 follows the tract through the tissue 1160 that was produced by biopsy device 1170 in FIG. 13A. Electrode 1220 is inserted with the spacer 1159 in place so that the distal end of the electrode 1220 stops substantially at the same location of the point of the deepest insertion of the biopsy device 1170 by means of successive engagement of the hub 1171 and hub 1222/1223 with spacer 1159. Then, RF output is delivered from generator 1100 to produce ablation zone 1361 in the tissue, and the electrode temperature T, impedance Z, and current I are displayed to the user graphically on generator display 1106 as shown in FIG. 11 (note that numeric display of these readings can also be included here as shown in FIG. 11). The generator provides the user intuitive information about the progress of the ablation formation 1316 by means of the graphs. When the temperature T rises and begins to stabilize at a desired level and/or the impedance Z drops and begins to stabilize at a level, this indicates to the user the ablation zone 1361 has formed substantially around the electrode tips 1225A, 1225B. Similarly, the graph of the output current I indicates to the user that the automatic controller of the generator requires less output level to maintain the desired set temperature, which is another indication of ablation formation. Increased tissue temperature around the tip tends to reduce the measured impedance Z because higher temperature produces higher ion mobility. This is a clear signal to the user that the electrode can start to be withdrawn to extend the ablation zone along the biopsy tract. In some embodiments, the instructions for use (IFU) and/or labeling of the electrode can instruct the user to rotate the electrode to prevent or break adhesion between the electrode and tissue due to tissue coagulation and thereby facilitate withdrawal; such instruction and rotation can be applied to any of the electrodes 1120, 1220, 1320, 1420. This is an important step in one method of the present invention. In some embodiments, the electrode tip(s) can be polished to high smoothness and/or silicon fluid to reduce adhesion between the electrode and the coagulated tissue; such processing can be applied to any of the electrodes 1120, 1220, 1320, 1420.

The user then withdraws the electrode 1220 through the introducer 1150. As shown on the screen 1106 in FIG. 13C, the generator 1100 displays a corresponding drop in temperature T and rise in impedance Z because the tips 1225A and 1225B have moved into cooler tissue. This is an important indication to the user to stop or slow withdrawal of the electrode 1220 until the ablation zone has time to form at the new location of the electrode tip(s). Without this indication, the user must rely more heavily or completely on the rate of withdrawal of the electrode 1220 in order to fully heat the length of the biopsy tract. Furthermore, such user display, particularly graphical display and display in relation to a minimum/maximum/historical reading values, can indicate to the user when the electrode has been withdrawn too fast or too far by showing a large and/or fast change in value; this has the advantage that the user can re-advanced the electrode into the tissue to close a potential gap. Having slowed or stopped the electrode 1220 withdrawal, the temperature T and impedance Z rise and drop, respectively, and begin to stabilize, as shown on the generator display 1106, as the ablation zone 1362 extends to encompass the new location of the tips 1225A, 1225B. Once this occurs, the physician user can continue to withdraw the electrode 1220 as described in the foregoing.

FIG. 13D shows the generator display 1106 and the position of the electrode 1220 after a number of such cycles of interrupted retraction of the electrode 1220. The electrode IFU and labeling can include user instruction to rotate the electrode at each such interruption of withdrawal, or even continuously, to prevent stickage between the electrode and coagulated tissue. FIG. 13D also shows the electrode about to enter the uninsulated shaft of the introducer 1150. As the proximal electrode tip 1225A enters the introducer 1150, the impedance Z and its graph will likely show a large drop because the proximal tip 1225A will have energized the introducer 1150. This is a useful indication to the user that the withdrawal in complete and the tract has been ablated up to the introducer.

FIG. 15 refers collectively to FIGS. 15A, 15B, 15C, and 15D. FIG. 15 shows the process of producing a tract burn after tissue biopsy in accordance with the present invention. FIG. 15 differs from FIG. 13 in two ways. First, in FIG. 13 both the biopsy device 1170 and the electrode 1220 were while the biopsy device 1170 was inserted through introducer 1150 using a spacer 1159. In FIG. 15, the spacer 1159 is not use with electrode 1220, so its distal point extends beyond the previous point of deepest insertion of the biopsy device, thereby further ensuring that the tract burn encompass the entire tract. This approach is facilitated by the inclusion of a sharp point on the electrode distal end as shown for electrode 1420 in FIG. 14. Second, in FIG. 13, the electrode 1220 is withdrawn through the introducer 1150. In FIG. 15, the electrode 1220 and the introducer 1150 are withdrawn as a unit; this has the advantage that it facilitates the ablation of a longer section of the tract of the biopsy device 1170, up to or beyond the distal end of the original position of the introducer 1150. The tract burn can continue further after the state shown in FIG. 15D, but is preferably stopped before the skin surface is reached in order to avoid skin burn. The impedance reading can be useful in this regard as the average impedance can change as the ablation electrode passes through different tissue types are approach the skin surface; this is an important safety feature of a tract burn system that include impedance monitoring and display to the user, particularly by means of a display that includes historical impedance information in addition to real-time impedance readings (such as line graph of impedance, a bar graph that caches the minimum and/or maximum impedances, or a numeric display of the minimum and/or maximum impedance). FIG. 15 demonstrates the process of controlling a tract burn by means of temperature and impedance displays, in addition to optional display of the generator output level (in this case, current), just as in FIG. 13.

The processes and generator display features of FIGS. 13 and 15 can apply to each of the ablation devices 1120, 1220, 1320, and 1420. The generator display features of FIGS. 11 and 12 can each be applied to FIGS. 13 and 15.

FIG. 14 shows several embodiments of ablation devices in accordance with the present invention. FIG. 14 shows biopsy device 1170, a bipolar electrode 1320, a monopolar electrode 1420, spacer 1159, and introducer 1150 separate from each other. Line 1400 shows how biopsy device 1170, bipolar electrode 1320, monopolar electrode 1420, spacer 1159 can each be inserted into introducer 1150. Biopsy device 1170, bipolar electrode 1320, and monopolar electrode 1420 can each be inserted into cannula 1150, but not at the same time. If spacer 1159 is coupled with the introducer 1150, then biopsy device 1170, bipolar electrode 1320, and monopolar electrode 1420 can each be insertion through both spacer 1159 and introducer 1150 one at a time.

Bipolar electrode 1320 includes generator cable 1321, hub 1322, coupling feature 1323, insulation shaft portion 1324A, a first bipolar active tip 1325A, a second shaft insulated portion 1324B, and a second bipolar active tip 1325B. Bipolar electrode 1320 has the same features as electrode 1220 with the addition that electrode 1320 has a protrusion 1326 form its distal point that houses a temperature sensor and holds the temperature sensor distal to the distal tip 1325B. The protrusion 1326 can be electrically insulated or connected to the tip 1325B. The protrusion 1326 can be a cylinder with smaller diameter than the tip 1325B, or it can be a conical or sharped point. The protrusion 1326 can be applied to electrodes 1120 and 1220. In embodiments wherein the electrode tip(s) of 1120, 1220, or 1320 is fluid cooled, protrusion 1326 can be used to monitored temperature while fluid-cooled ablation is performed.

Monopolar electrode 1420 includes generator cable 1421, hub 1422, coupling feature 1423, insulation shaft portion 1424, active tip 1425. Electrode 1420 has the same features as electrode 1120, except that electrode 1420 has sharpened distal point. A sharpened distal point can be applied to electrodes 1120 and 1220. A sharpened point has the advantage that the ablation electrode can penetrate tissue, including tissue beyond the tissue tract produced by a coaxial biopsy device 1170.

The biopsy device 1170 and the introducer needle 1150 can be of the type commonly used in the medicine, for example, the Temno coaxial biopsy devices and introducer needles marketed by Carefusion. Such introducers 1150 typically include a shaft 1154 that is an ultra-thin-wall hypotube, such as sizes 20UTW, 19UTW, 17UTW, 15UTW, 13.5UTW, which are compatible with biopsy device whose outer shafts are 22G, 20G, 18G, 16G, 14G, respectively. The introducer is also provided with a stylet for its initial insertion into the tissue. The biopsy devices 1170 are configured to pass through the introducer 1150 with small clearance, e.g. 0.1001″ to 0.10015″. It is an advantage of the present invention that the outer diameter of the electrode (e.g. 1120, 1220, 1320, 1420) is configured to be compatible with commonly available biopsy introducer introducers and to have tight tolerance with the inner diameter of said introducer's lumen that is similar to the tolerance of commonly available biopsy devices, e.g. 1170. Such tight tolerance to the inner diameter of the introducer 1150 for both the outer diameter of the biopsy device 1170 and the outer diameter of the electrode (1120, 1220, 1320, or 1420) helps ensure that the electrode will follow the same tract in the tissue 1160 and thereby ensure that tissue touched/cut by the biopsy device 1170 is reliably coagulated by the electrode. Such compatibility requires special dimensioning of the outer diameter of maximum outer diameter portion of the electrode shaft, which is commonly the most proximal insulated portion (e.g. 1124 of electrode 1120, 1224A of electrode 1220). Such compatibility can be enabled by carefully stacking up the dimensions of hypodermic tubing and plastic insulation used to construct the electrodes, such as electrodes 1120, 1220, 1320, 1420. Configuration of the maximum outer diameter of tract burn electrodes, such as 1120, 1220, 1320, and 1420, to match the outer diameter of commonly available biopsy devices 1170.

It is understood that the display features of generator 1100 are applicable to tract burns produced by other medical probes, including the tract produced by an ablation electrode (e.g. 1120, 1220, 1320, 1420) itself. The graphical display of both the temperature and impedance on the same time axis gives complementary information to the user to help control the withdrawal of an ablation electrode to produce a confluent, uninterrupted tract burn. Because the temperature measurement is at a single location, it provides precise information about temperature at one location along the electrode active tip, but does not provide complete information about the heating around the entire electrode tip. On the other hand, Impedance (and impedance changes in particular) provides integrated information about heating along the electrode active tip, but does not provide precise information about tissue temperature. As such, temperature and impedance provide different, complementary information to the user during a tract burn. Displaying these two readings, and in particular graphing them, at the same time along with the generator output level provides rich information that the user can use to avoid gaps in the tract ablation zone.

It is typical to place the temperature sensor of an electrode at the distal end of the shaft. This is advantage because it gives information that the most distal part of the tract is ablated. For a monopolar electrode, the distal tip is the point of maximum temperature; however, for a bipolar electrode, the maximum temperature can be between the tips (i.e. in the insulated region between the tips, e.g. 1224B of electrode 1220). As such, the temperature may go up when a user withdraws a bipolar electrode during tract burn. This can give a false sense of completeness of the ablation zone because the actual maximum heating is occurring proximal to this location, at the location between the active tips (e.g. 1224B). However, the impedance will tend to go down in this situation because the proximal tip has entered cooler tissue. This is a special advantage of displaying both impedance and temperature for a bipolar electrode used for tract burn.

Time graphing of temperature and impedance on the same time axis as shown in FIG. 11 has special advantage in that it shows the full time history of these readings, so that the physician user can review them at a glance if the user's attention needs to turn away from the screen at some point during the electrode withdrawal. The shape of these time graphs is also useful itself because it shows the rate of change of these values in addition to the magnitude of the change. For example, a large, fast change in one or both of these values can indicate to user that the electrode has been withdrawn too quickly or too far, and thus that the electrode may need to be re-advanced into the tissue to close a gap in the ablation zone.

Displaying the minimum impedance during a tract burn (as shown by line 1212B in FIG. 12, or as shown by the impedance graph 1112A in FIG. 11) is a special advantage of the present invention because impedances are generally interpreted as relative not absolute values. The absolute value of an impedance is confounded by numerous factors unrelated to tissue heating, such as the tissue type around the electrode tip or the distance between a monopolar electrode tip and a ground pad. As such, for tract burn, observation of the change in impedance is most relevant to assessing completion of the ablation at the current electrode position. It an advantage for a physician to be able to refer back to the minimum impedance measured as a reference for sufficiently heated tissue for the particular electrode, tissue, and system configuration of a given tract burn. Without such reference, the physician must remember this minimum value, and doing so is difficult when attending to multiple other thing during a dynamic track burn procedure.

In another embodiment, the ablation electrodes 1120, 1220, 1320, 1420, can each include more than one temperature sensor at multiple location along the shaft, and the temperature of these multiple temperature sensors can be displayed numerically and graphically on generator 1100 at the same time as shown in FIGS. 11 and 12, including plotting more than one temperature on the same time axis. For example, temperature sensors can be located at the distal and proximal end of an electrode tip(s), and/or at the midpoint of the tip (for example at the location of the distal insulation 1224B of a bipolar electrode). Such multiple temperature sensors and simultaneous display can give the user additional information about the geometry of heating around the electrode tip(s), and thus better control over the tract ablation process.

The display of temperature of a biopsy tract coagulation electrode per se is an aspect of the present invention and is useful because it directly measures tissue heating and can be used to control the movement of the electrode and ensure a continuous tract coagulation. The display of impedance of a biopsy tract coagulation electrode per se is useful because it provides the user information about tissue heating around the ablation tip which can be used to control the movement of the electrode and ensure a continuous tract coagulation. The combined display of temperature and impedance of a biopsy tract coagulation electrode provides improved information about heating around the electrode and can be used to further improve movement of the electrode and ensure a continuous tract coagulation.

Monopolar tract ablation has the advantage of producing a more extended ablation zone along the active tip, but requires the use of a ground pad. Bipolar tract ablation has the advantage not requiring a ground pad, but produces a more focused and smaller ablation between the active tips that can require more controlled electrode withdrawal.

Electrodes (such as 1120, 1220, 1320, and 1420) can be constructed in a number of ways in accordance with the present invention. In one embodiment, these electrodes can be produced by coaxial nesting of metal tubing (e.g. stainless steel hypotube) and plastic tubing (e.g. polyimide, polyester, Teflon). Monopolar electrodes, such as 1120 and 1420, can be constructed from a hypotube over which a plastic tube is applied to form the insulated portions 1124 and 1424, respectively, leaving the distal active tips 1125 and 1425, respectively, as uninsulated hypotube. Bipolar electrodes such as 1220 and 1320, can be formed from an inner hypotube whose uncovered distal end forms the distal active tip 1225B, 1325B, respectively; overlaying that coaxially by a plastic tube whose distal end forms inter-tip insulated region 1224B, 1324B, respectively; overlaying that coaxially by a hypotube tube whose distal end forms proximal active tip 1225A, 1325A, respectively; and overlaying that coaxially by a plastic tube whose distal end forms the proximal insulated shaft portion 1224A, 1324A, respectively. In other embodiments, the active tips of electrodes (e.g. 1120, 1220, 1320, 1420) can be formed by applying cylindrical electrodes over a primarily insulated central shaft. In other embodiment, electrodes (e.g. 1120, 1220, 1320, and 1420) can be constructed in other way as are known to one skilled in the art.

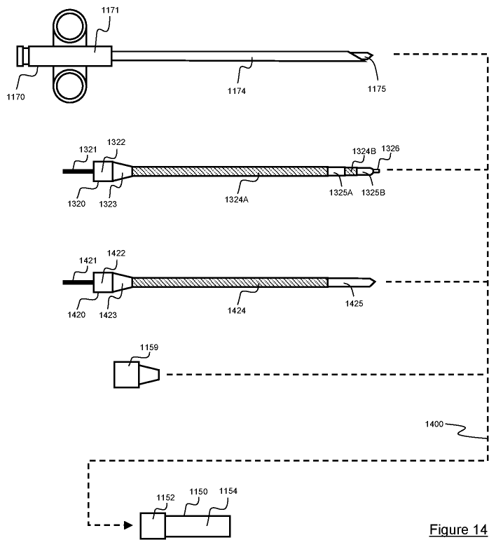

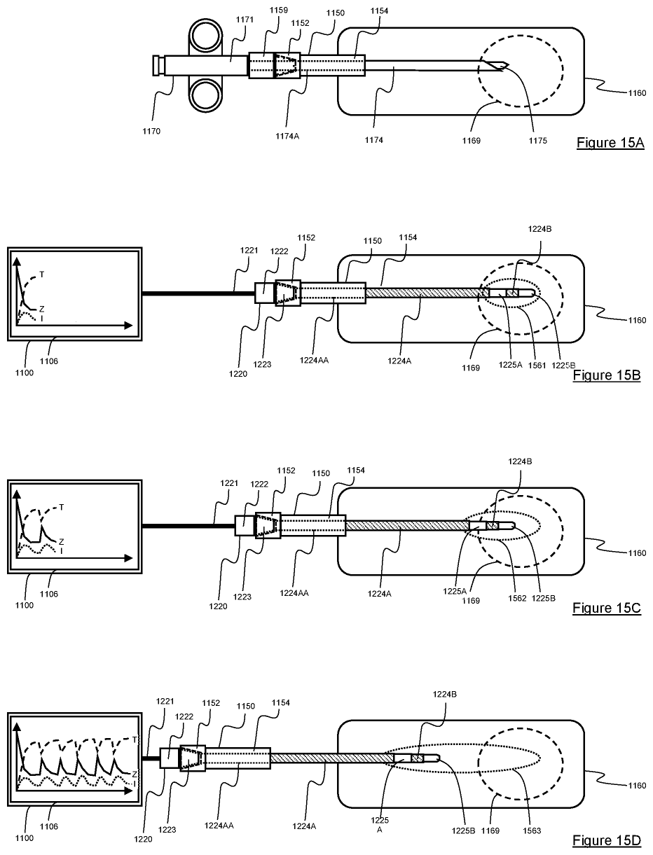

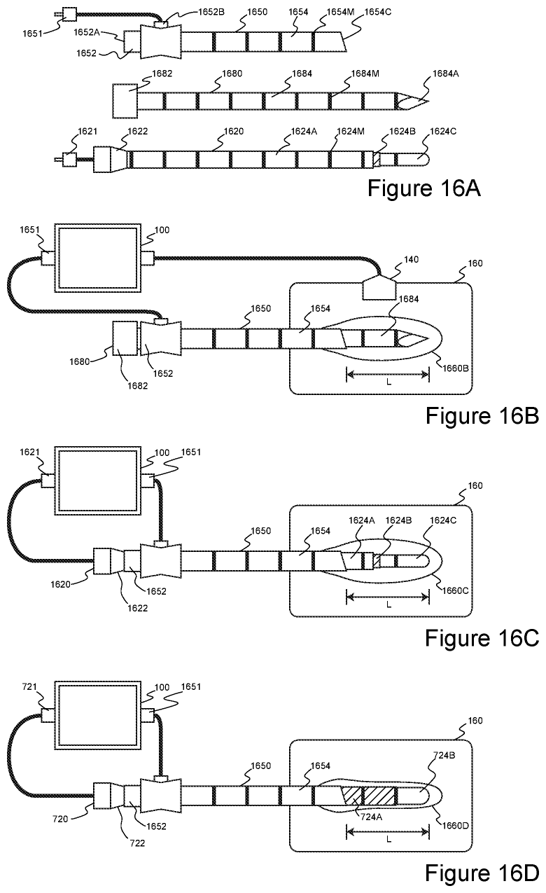

FIG. 16 refers collectively to FIGS. 16A, 16B, 16C, and 16D.

In FIG. 16A, the cannula 650 in the system of FIG. 6A is replaced by cannula 1650, which includes a connection 1652B to one output pole of an electrosurgical generator, such as generator 100, via cable 1651. As such, cannula 1650 provides both for probe-insertion (e.g. biopsy needle) and electrode functions. Cable 1651 can be integrally connected to the cannula 1650, or it can disconnectable from jack 1652B, which disconnection has the advantage of facilitating use of cannula 1650 to insert a biopsy needle 670 to a precise location without perturbation by a generator cable that can be heavy or can get tangled. Connection 1651 is conductively connected to the electrically conductive, non-insulated, cannula shaft 1654. Cannula 1650 includes hub 1652 at it proximal end, shaft 1654 at is distal end, shaft depth markers 1654M, and a lumen through its length from proximal hub opening 1652A to distal shaft opening 1654C, through which biopsy needle 670 can be inserted into tissue. The dimensions of cannulas 1650 and 650 can be substantially similar in relation to their use as insertion devices for a biopsy device 670, including in relation to the biopsy device's extension length L. In addition, FIG. 16A shows an elongated stylet 1680 having a proximal end cap 1682, elongated electrically conductive shaft 1684, depth markers 1684M, and a sharp distal trocar point 1684A. In additional, FIG. 16A shows a monopolar electrode 1620 that includes a proximal hub 1622 and a proximal electrically conductive shaft 1624A that is electrically isolated from, and separated by electrically insulated shaft portion 1624B from, its conductive active tip 1624C which is conductively connected to connector 1621 that connects to one pole of an electrosurgical generator.

FIG. 16B shows electrically passive but conductive stylet 1680 inserted into cannula 1650, and the combination of the probe 1680 and cannula 1650 used for form ablation zone 1660B in tissue 160 in a monopolar configuration. Generator 100 conducts an electrical signal via connection 1651 to the cannula shaft 1654, which in turns conducts the signal to the conductive stylet shaft 1684, thereby forming a unified active tip 1654, 1684 around which the ablation zone 1660B forms. In some embodiments, the stylet 1680 can have blunt distal point like 1784A, which has the advantage of tending to follow the tissue tract formed by prior piercing insertion of a biopsy device 670 via cannula 1650, rather than forging a new path through the tissue (which would be more likely with a sharp point 1684A). In some embodiments, the stylet 1680 can be a biopsy needle (such as needle 670); this has the advantage of reducing the number of parts in a biopsy track burn kit and increases the likelihood of coagulating more exactly the tissue tract that was formed earlier insertion of the biopsy needle. It is an advantage that a biopsy introducer cannula, such as 1650 and 1750, that has an electrical connection to an electrosurgical generator because it can remove the need for a separate electrode, or a special electrified biopsy device, to perform ablation after biopsy.

FIG. 16C shows monopolar electrode 1620 with passive conductive proximal shaft 1624A inserted into tissue 160 via cannula 1650, and the combination of electrode 1620 and cannula 1650 used for bipolar ablation to form coagulation zone 1660C. One potential of generator 100 is conducted to electrode distal active tip 1624C via cable 1621, and the other potential of generator 100 is conducted to conductive cannula shaft 1654 via cable 1651 and then conducted to proximal conductive electrode shaft portion 1624A via its conductive contact with the conductive inner lumen of cannula shaft 1654.

FIG. 16D shows monopolar electrode 720 with electrically-insulated proximal shaft 724A and conductive distal active tip 724B inserted into tissue 160 via cannula 1650, and the combination of electrode 720 and cannula 1650 used for bipolar ablation to form coagulation zone 1660D. One potential of generator 100 is conducted to electrode distal active tip 724B via cable 721, and the other potential of generator 100 is conducted to conductive cannula shaft 1654 via cable 1651. Electrode shaft insulation 724A prevents short circuiting between the active contacts 1654 and 724B of the combined bipolar probe, composed of electrode 720 and cannula 1650, unless then electrode active tip 724B is withdrawn into cannula shaft 1654.

Referring to FIGS. 16B-16D, the extension length L for each probe 1680, 1620, 720 is substantially similar to that for biopsy needle 670 when inserted through cannula 1650. Though the cannula shaft 1654 can be wholly electrified by contact between cannula lumen and the electrode shaft (which occurs in the configuration of FIG. 10D if electrode active tip 724B is withdrawn into cannula shaft 1654), ablation zone (1660B, 1660, 1660D) forms primarily at the distal end of the combined shaft of the electrode and cannula due to the geometry of the electric field being highest at the unified probe's distal point. However, there is risk of skin burn at the surface of tissue 160 due to elevated electric fields at that location as well as along the entirety of the cannula shaft 1654, particularly if the depth of insertion of the cannula 1650 into the tissue 160 is shallow. An advantage of an uninsulated cannula 1650 is that is more simple, less expensive, and more readily available than an insulated cannula (such as 1750); however, a partially insulated cannula having an electrically non-conductive proximal shaft portion (e.g. 1750) has the advantage of reducing the risk of thermal damage to non-target structures (such as the skin, organs, and biological divisions such as the chest wall) along the proximal length of the cannula shaft and allows the physician both to control thermal damage to more superficial non-target structures during ablation and to ablate tissue around the distal opening of the cannula (e.g. 1754C).

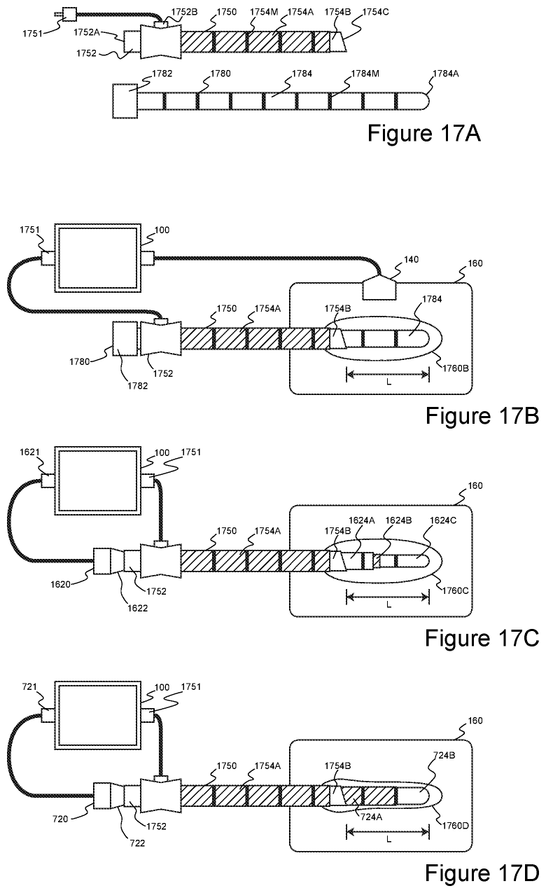

FIG. 17 refers collectively to FIGS. 17A, 17B, 17C, and 17D.

In FIG. 17A, the cannula 650 in the system of FIG. 6A is replaced by cannula 1750, which includes a connection 1752B to one output pole of an electrosurgical generator, such as generator 100, via cable 1751. As such, cannula 1750 provides both for probe-insertion (e.g. biopsy needle) and electrode functions. Cable 1751 can be integrally connected to the cannula 1750, or it can disconnectable from jack 1752B, which disconnection has the advantage of facilitating use of cannula 1750 to insert a biopsy needle 670 to a precise location without perturbation by a generator cable that can be heavy or can get tangled. Connection 1751 is conductively connected to the electrically conductive active tip 1754B of the cannula shaft. The remainder of the shaft 1754A, its proximal end, is electrically non-conductive. Cannula 1750 includes hub 1752 at its proximal end, shaft 1754A and 1754B at is distal end, shaft depth markers 1754M, and a lumen through its length from proximal hub opening 1752A to distal shaft opening 1754C, through which biopsy needle 670 can be inserted into tissue. The dimensions of cannulas 1750 and 650 can be substantially similar in relation to their use as insertion devices for a biopsy device 670, including in relation to the biopsy device's extension length L. In addition, FIG. 17A shows an elongated stylet 1780 having a proximal end hub 1782, elongated electrically conductive shaft 1784, depth markers 1784M, and blunt distal point 1784A.

FIG. 17B shows electrically passive but conductive stylet 1780 inserted into cannula 1750, and the combination of the probe 1780 and cannula 1750 used for form ablation zone 1760B in tissue 160 in a monopolar configuration. Generator 100 conducts an electrical signal via connection 1751 to the cannula shaft active tip 1754B, which in turns conducts the signal to the conductive stylet shaft 1784, thereby forming a unified active tip 1754B, 1784 around which the ablation zone 1760B forms. In some embodiments, the stylet 1680 can have sharp distal point like stylet 1684A, which has the advantage of facilitating penetration of tissue. However, a blunt point 1784A has the advantage of tending to follow the tissue tract formed by prior piercing insertion of a biopsy device 670 via cannula 1750, rather than forging a new path through the tissue (which would be more likely with a sharp point like 1684A). In some embodiments, the stylet 1780 can be a biopsy needle (such as needle 670); this has the advantage of reducing the number of parts in a biopsy track burn kit (which can be an important clinical, practical, competitive advantage) and increases the likelihood of coagulating more exactly the tissue tract that was formed earlier insertion of the biopsy needle. It is an advantage that a biopsy introducer cannula, such as 1650 and 1750, that has an electrical connection to an electrosurgical generator because it can remove the need for a separate electrode, or a special electrified biopsy device, to perform ablation after biopsy.

FIG. 17C shows monopolar electrode 1620 with passive conductive proximal shaft 1624A inserted into tissue 160 via cannula 1750, and the combination of electrode 1620 and cannula 1750 used for bipolar ablation to form coagulation zone 1760C. One potential of generator 100 is conducted to electrode distal active tip 1624C via cable 1621, and the other potential of generator 100 is conducted to conductive cannula shaft 1754B via cable 1751 and then conducted to proximal conductive electrode shaft portion 1624A via its conductive contact with the conductive inner lumen of cannula shaft 1654. As such, cannula active tip 1754B and electrode proximal conductive shaft 1624A form a combined active tip 1754B, 1624A.

FIG. 17D shows monopolar electrode 720 with electrically-insulated proximal shaft 724A and conductive distal active tip 724B inserted into tissue 160 via cannula 1750, and the combination of electrode 720 and cannula 1750 used for bipolar ablation to form coagulation zone 1760D. One potential of generator 100 is conducted to electrode distal active tip 724B via cable 721, and the other potential of generator 100 is conducted to conductive cannula shaft 1754B via cable connection 1751. Electrode shaft insulation 724A prevents short circuiting between the active contacts 1654 and 724B of the combined bipolar probe, composed of electrode 720 and cannula 1670, unless then electrode active tip 724B is withdrawn into cannula active tip 1754A, in which case a short circuit (i.e. a very low impedance between the generator output poles) can signal to the generator 100 or its user that the electrode 720 has been withdrawn into the cannula 1750.

Referring to FIGS. 17B-17D, the extension length L for each probe 1780, 1620, 720 is substantially similar to that for biopsy needle 670 when inserted through cannula 1750. A partially insulated cannula having an electrically non-conductive proximal shaft portion (e.g. 1750, 650) has the advantage of reducing the risk of thermal damage to non-target structures (such as the skin, organs, and biological divisions such as the chest wall) along the proximal length of the cannula shaft and allows the physician both to control thermal damage to more superficial non-target structures during ablation and to ablate tissue around the distal opening of the cannula (e.g. 1754C, 654C).

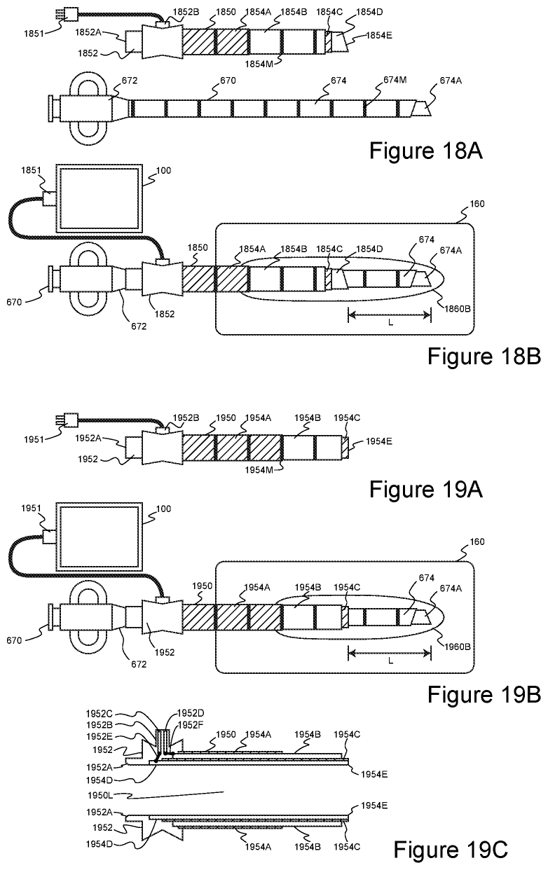

FIG. 18 refers collectively to FIGS. 18A and 18B.

In FIG. 18A, the cannula 650 in the system of FIG. 6A is replaced by cannula 1850, which includes a connector 1852B to both output poles of an electrosurgical generator, such as generator 100, via cable 1851. As such, cannula 1850 provides both for probe-insertion (e.g. biopsy needle) and bipolar-electrode functions. Cable 1851 can be integrally connected to the cannula 1850, or it can disconnectable from jack 1852B, which disconnection has the advantage of facilitating use of cannula 1850 to insert a biopsy needle 670 to a precise location without perturbation by a generator cable that can be heavy or can get tangled. A first lead of connection 1851, 1852B is conductively connected to the distal electrically conductive active tip 1854D of the cannula shaft. The second lead of connection 1851, 1852B is conductively connected to the proximal electrically conductive active tip 1854B of the cannula shaft. The two active tips 1854B, 1854D are electrically insulated and separated from each other by non-conductive inter-tip shaft portion 1854C. The remainder of the cannula shaft 1854A, its proximal end, is electrically non-conductive. Cannula 1850 includes hub 1852 at its proximal end; shaft 1854A, 1854B, 1854C, 1854D at its distal end; shaft depth markers 1854M; and a lumen through its length from proximal hub opening 1852A to distal shaft opening 1854E, through which biopsy needle 670 can be inserted into tissue. The dimensions of cannulas 1850 and 650 can be substantially similar in relation to their use as insertion devices for a biopsy device 670, including in relation to the biopsy device's extension length L. In addition, FIG. 18A shows biopsy device 670 having shaft 674, which includes its distal shaft point 674A.

FIG. 18B shows electrically passive but conductive biopsy needle 670 inserted into cannula 1850, and the combination of the probe 670 and cannula 1850 used for form ablation zone 1860B in tissue 160 in a bipolar configuration. Generator 100 conducts a first electrical signal (e.g. a radiofrequency signal) via connection 1851 to the cannula shaft distal active tip 1854D, which in turns conducts the signal to the conductive biopsy shaft 674 by conductive contact between the biopsy shaft 674 and the inner lumen of the electrode distal active tip 1854D, thereby forming a unified active tip 1854D, 674. Generator 100 conducts a second electrical signal (e.g. a reference potential) via connection 1851 to the cannula shaft active tip 1854B. The combined distal tip 1854D, 674 and the proximal tip 1854B form a bipolar pair around which the ablation zone 1860B forms. One advantage of this bipolar cannula 1850 is that it avoids the need for a separate electrode that matches the biopsy device 670, thereby reducing the number of parts in a biopsy track burn kit (which can be an important clinical, practical, competitive advantage) and increases the likelihood of coagulating more exactly the tissue tract that was formed earlier insertion of the biopsy needle. It is a further advantage that the cannula 1850 can be used with biopsy devices 670 having various different lengths (e.g. from different manufacturers) and the tissue tract formed by each can be coagulated more accurately because the same device used for biopsy is later used for biopsy tract burn. In some embodiments, an extended stylet, such as 1680 or 1780, can be used for ablation with cannula 1850 instead of the biopsy device 670.

FIG. 19 refers collectively to FIGS. 19A and 19B.

In FIG. 19A, the cannula 650 in the system of FIG. 6A is replaced by cannula 1950, which includes a connector 1952B to both output poles of an electrosurgical generator, such as generator 100, via cable 1951. As such, cannula 1950 provides both for probe-insertion (e.g. biopsy needle) and bipolar-electrode functions. Cable 1951 can be integrally connected to the cannula 1950, or it can disconnectable from jack 1952B, which disconnection has the advantage of facilitating use of cannula 1950 to insert a biopsy needle 670 to a precise location without perturbation by a generator cable that can be heavy or can get tangled. A first lead of connection 1951, 1952B is conductively connected to the distal electrically conductive active tip 1954D of the cannula shaft, which is shrouded by inter-tip insulation region 1954C but visible in the cross-sectional view of FIG. 19C. The second lead of connection 1951, 1952B is conductively connected to the proximal electrically conductive active tip 1954B of the cannula shaft. The two active tips 1954B, 1954D are electrically insulated and separated from each other by non-conductive inter-tip shaft portion 1954C. The remainder of the cannula shaft 1954A, its proximal end, is electrically non-conductive. Cannula 1950 includes hub 18952 at its proximal end; shaft 1954A, 1954B, 1954C, 1954D at its distal end; shaft depth markers 1954M; and a lumen through its length from proximal hub opening 1952A to distal shaft opening 1954E, through which biopsy needle 670 can be inserted into tissue. The dimensions of cannulas 1950 and 650 can be substantially similar in relation to their use as insertion devices for a biopsy device 670, including in relation to the biopsy device's extension length L.

FIG. 19B shows electrically passive but conductive biopsy needle 670 inserted into cannula 1950, and the combination of the probe 670 and cannula 1950 used for form ablation zone 1960B in tissue 160 in a bipolar configuration. Generator 100 conducts a first electrical signal (e.g. a radiofrequency signal) via connection 1951 to the cannula shaft shrouded active tip 1954D, which in turns conducts the signal to the conductive biopsy shaft 674 by conductive contact between the biopsy shaft 674 and the inner lumen of the electrode distal active tip 1954D, thereby forming a distal active tip that is primarily comprised of the portion of the biopsy needle shaft 674 that protrudes from the cannula distal opening 1954E. Generator 100 conducts a second electrical signal (e.g. a reference potential) via connection 1951 to the cannula shaft active tip 1954B. The distal tip 674 and the proximal tip 1954B form a bipolar pair around which the ablation zone 1960B forms. One advantage of this bipolar cannula 1950 is that it avoids the need for a separate electrode that matches the biopsy device 670, thereby reducing the number of parts in a biopsy track burn kit (which can be an important clinical, practical, competitive advantage) and increases the likelihood of coagulating more exactly the tissue tract that was formed earlier insertion of the biopsy needle. It is a further advantage that the cannula 1950 can be used with biopsy devices 670 having various different lengths (e.g. from different manufacturers) and the tissue tract formed by each can be coagulated more accurately because the same device used for biopsy is later used for biopsy tract burn. In some embodiments, an extended stylet, such as 1680 or 1780, can be used for ablation with cannula 1950 instead of the biopsy device 670.

FIG. 19C shows a schematic cross section of the bipolar cannula 1950 along its length. As the cannula structure is roughly tubular, some cannula parts appear as two separate objects in the cross-sectional figure. The shrouded active tip 1954D forms the walls of the inner lumen of the shaft of the cannula 1950. Connector 1952B includes a first socket 1952C that connects to a first generator potential and to the shrouded active tip 1954D via conductive connection 1952E within the cannula hub 1952. Connector 1952B includes a second socket 1952D that connects to a second generator potential and to the proximal active tip 1954B via conductive connection 1952F within the cannula hub 1952. In some embodiments, the alternation of conductive and non-conductive tubes shown for cannula 1950 in FIG. 19C can be used for the construction of other cannulas and electrode having conductive and non-conductive portions, such as 650, 720, 820, 920, 1620, and 1850.

In some embodiments of the present invention, such as those shown in FIGS. 1A through 19C, the biopsy device can be withdrawn into the cannula (e.g. 150, 550, 650) after a tissue sample is collected, and the cannula (e.g. 150, 550, 650) redirected in the tissue, but still be positioned in the same tissue tract, and the biopsy device reinserted into a new direction to collect a different sample. After each such sample collection, and before the cannula is moved or redirected, the tissue tract and cells at the distal end of cannula opening of the cannula (e.g. 154C, 554C, 654C, respectively) can be ablated.

It is understood that the features of the present invention can be combined with different types of biopsy devices, including without limitation, core biopsy devices, fine-needle aspiration needles, spring-loaded devices, manual devices.

In other embodiments, the ablation electrodes (120, 220, 320, 520, 620, 720, 820, 920, 1120, 1220, 1320, 1420) and cannula (150, 550, 650, 1050, 1650, 1750, 1850, 1950) can each include more than one temperature sensor at multiple location along the shaft, and the temperature of these multiple temperature sensors can be displayed numerically and graphically on generator 100 at the same time (for example as shown in FIGS. 1, 2, 4, and 5), including plotting more than one temperature on the same time axis. For example, temperature sensors can be located at the distal and proximal end of an electrode tip(s), and/or at the midpoint of the tip (for example at the location of the distal insulation 225A of a bipolar electrode). Such multiple temperature sensors and simultaneous display can give the user additional information about the geometry of heating around the electrode tip(s), and thus better control over the tract ablation process.

It can be a significant advantage that partially-insulated cannula 150, 550, 650, 1650, 1750, 1850, 1950 can be made available in multiplicity which is characterized by variation in the cannula tip length (e.g. 154B, 554B, 654B, 1754B, 1854B, 1854D, 1954B) to adapt to different clinical applications and methods and tumor/target sizes, and/or which is characterized by variation in the cannula shaft (e.g. 154) length to produce different lengths of extension of the biopsy device (e.g. 170 or 120) and electrode (e.g. 320 and 120) beyond the cannula distal end (e.g. 154C) to suit different clinical applications and methods and tumor/target sizes.

It is an advantage that a single kit, such as those shown in FIG. 1A-19C, can be used for both tract burn (as shown in FIG. 1A) and tumor ablation (as shown in FIG. 1B).

In some embodiments, each of the electrodes 120, 220, 320, 520 620, 720, 820, 920, 1120, 1220, 1320,1420, 1620 can omit or include one or more temperature sensors; can omit or include fluid channels configured for cooling the electrode shaft during energy delivery; can omit or include outflow holes on the electrode shaft configured for perfusion during energy delivery; can include a generator-connection cable that is integrated into the electrode or a connector for a generator-connection cable; can include fluid tubing that is integrated into the electrode or connector(s) for connection to a fluid pump, source, and sink.

In some embodiments, the electrode features (e.g. insulation, active tip(s)) of each electrode 320, 620, 720, 820, 920, 1120, 1220, 1320,1420, 1620 can be integrated into a biopsy device (such as 670) to create a combined electrode-biopsy device (such as 120, 220, 520). This can be an advantage because it reduces the number of components in a kit by integrating the biopsy and electrode features and functions into a single device and ensures that the biopsy tract is accurately coagulated because the same device is used for biopsy and for coagulation.

In some embodiments, electrode and cannula are withdrawn as a unit to coagulate the tissue tract formed by a biopsy device that had been inserted via and cannula, as well as some or all of the tissue tract formed by the cannula itself. In some embodiments, the electrode is withdrawn into the cannula to coagulate the tissue tract formed by a biopsy device that had been inserted via and cannula and to coagulate tissue around the distal opening of the cannula from which the biopsy extended from the cannula into the tissue. In some embodiments, the electrode is withdrawn into the cannula to coagulate the tissue tract formed by a biopsy device that had been inserted via and cannula and to coagulate tissue around the distal opening of the cannula from which the biopsy extended from the cannula into the tissue; and then the electrode and cannula are withdrawn as a unit to coagulate some of all of the remainder of the tissue tract formed by the cannula itself. In some embodiments, each of the three technique of the previous sentences of this paragraph can be utilized with any of the systems presented in this application, including those shown in FIGS. 1A through 19C.

Other embodiments are within the scope of the following claims.

What is claimed is:

1. A system including a cannula and an electrode, wherein the cannula is configured to introduce a biopsy needle into bodily tissue, the cannula is configured to introduce the electrode into the bodily tissue, the cannula shaft includes an electrically conductive shaft portion, the electrode shaft includes an electrically conductive shaft portion, the combination of the cannula and electrode is configured to operate with an electrosurgical generator to ablate tissue around a tract through the tissue formed by prior introduction of the biopsy needle through the cannula and around the cannula distal end.

2. The system of claim 1, wherein the cannula shaft includes an electrically insulated portion at its proximal end, the cannula shaft includes an electrically conductive portion at its distal end around which tissue is ablated.

3. The system of claim 1, wherein the biopsy needle includes the electrode.

4. The system of claim 1, wherein the electrode and the biopsy needle protrude into the tissue beyond the cannula by the same length when each of the electrode and the biopsy needle are introduced by the cannula.

5. The system of claim 1, wherein the electrode is monopolar and cannula conductive shaft portion is energized by the electrode when the electrode introduced into the tissue via the cannula.

6. The system of claim 1, wherein the electrode is bipolar and cannula conductive shaft portion is energized by one of the electrodes contacts when the electrode when the electrode introduced into the tissue via the cannula.

7. The system of claim 1, wherein the electrode is bipolar electrode.

8. The system of claim 7, wherein the distal contact of the electrode has smaller surface area than does the proximal contact of the electrode.

9. The system of claim 2, wherein the electrode is a bipolar electrode, wherein the distal contact of the electrode has smaller surface area than does the proximal contact of the electrode, and wherein the cannula shaft conductive portion has smaller surface area than the distal contact of the electrode.

10. The system of claim 1, and further including a stylet, wherein the cannula includes the electrode, the cannula is configured to introduce the stylet into bodily tissue, and tissue is ablated around the stylet.
